(19) United States
(12) Patent Application Publication
(10) Pub. No.: US 2011/0245496 A1
(43) Pub. Date: Oct. 6, 2011

(54) Quinazoline Salt Compounds

(76) Inventors: Andrew Simon Craig, Kent (GB); David Malcolm Crowe, Kent (GB); Tim Chien Ting Ho, Kent (GB); Michael S. McClure, Durham, NC (US)

(21) Appl. No.: 12/663,337

(22) PCT Filed: Jun. 9, 2008
(86) PCT No.: PCT/US08/66254

§ 371 Date: Dec. 7, 2009

Related U.S. Application Data
(60) Provisional application No. 60/943,126, filed on Jun. 11, 2007.

(51) Int. Cl. C07D 405/02

(52) U.S. Cl. 544/293

(57) ABSTRACT

Salts of 4-quinazolineamines are described as well as methods of using the same in the treatment of disorders characterized by aberrant erbB family PTK activity.

### FIELD OF THE INVENTION

The present invention relates to quinazoline salt compounds as well as non-solvated or solvated forms thereof. In particular, the invention relates to salts of 4-quinazolineamines. These compounds are inhibitors of various protein tyrosine kinases (PTKs) of the erbB family and consequently are useful in the treatment of disorders mediated by aberrant activity of such kinases.

### BACKGROUND OF THE INVENTION

PTKs catalyze the phosphorylation of specific tyrosyl residues in various proteins involved in the regulation of cell growth and differentiation. (A. F. Wilks, Progress in Growth Factor Research, 1990, 2, 97-111; S. A. Courtneidge, Dev. Supp.l, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401). Inappropriate or uncontrolled activation of many PTKs, i.e. aberrant PTK activity, for example by over-expression or mutation, has been shown to result in uncontrolled cell growth.

Aberrant protein tyrosine kinase (PTK) activity has been implicated in a variety of disorders including psoriasis, rheumatoid arthritis, bronchitis, as well as cancer. Development of effective treatments for such disorders is a constant and ongoing enterprise in the medical field. The erbB family of PTKs, which includes c-erbB-2, EGFr, and erbB-4, is one group of PTKs that has attracted interest as a therapeutic target. Currently, of special interest, is the role of erbB family PTKs in hyperproliferative disorders, particularly human malignancies. Elevated EGFr activity has, for example, been implicated in non-small cell lung, bladder, and head and neck cancers. Furthermore, increased c-erbB-2 activity has been implicated in breast, ovarian, gastric and pancreatic cancers. Consequently, inhibition of erbB family PTKs should provide a treatment for disorders characterized by aberrant erbB family PTK activity. The biological role of erbB family PTKs and their implication in various disease states is discussed, for instance in U.S. Pat. No. 5,773,476; International Patent Application WO 99/35146; M. C. Hung et al, Seminars in Oncology, 26: 4, Suppl. 12 Aug. 1999, 51-59; Ullrich et al, Cell, 61: 203-212, Apr. 20, 1990; Modjtahedi et al, Intl J. of Oncology, 13: 335-342, 1998; and J. R. Woodburn, Pharmacol. Ther., 82: 2-3, 241-250, 1999.

International Patent Application PCT/EP99/00048 filed Jan. 8, 1999, and published as WO 99/35146 on Jul. 15, 1999, discusses PTKs including erbB family PTKs. This published application discloses bicyclic heteroaromatic compounds, including N-{3-Chloro-4-[(3-fluorobenzyl)oxy]phenyl}-6-[5-({[2-(methanesulphonyl)ethyl]amino}methyl)-2-furyl]-4-quinazolinamine. Also disclosed are hydrochloride salts of various 4-quinazolinamines. These compounds show inhibition activity against erbB family PTKs. Ditosylate salts of N-{3-Chloro-4-[(3-fluorobenzyl)oxy]phenyl}-6-[5-({[2-(methanesulphonyl)ethyl]amino}methyl)-2-furyl]-4-quinazolinamine are disclosed in International Patent Application No. PCT/US01/20706, filed Jun. 28, 2001, and published as WO 02/02552 on Jan. 10, 2002. The ditosylate salts of International Patent Application WO 02/02552 may be prepared in crystalline form and possess good moisture sorption properties (low hygroscopicity) and good physical stability.

One important property associated with solid state forms of drug substances is their aqueous solubility. Compounds having poor water solubility can lead to limited oral bioavailability when administered in patients. In such cases where the solubility of the drug substance is too low to allow the dose administered to dissolve in the patients intestinal volume then the compound is described as exhibiting solubility limited absorption. In such cases solid state forms with better aqueous solubility provide a significant opportunity to increase oral bioavailability and hence reduce the dosage required to be administered to the patient. In addition to reducing the dose burden to the patient, manufacturing costs for the drug product may be reduced. Furthermore solubility of drug substances in aqueous systems is a crucial factor for designing drug products for parenteral and transdermal administration as the drug substance must be applied in solution. Accordingly, compounds having aqueous solubility which approaches optimal values is an constant goal in the pharmaceutical field.

### SUMMARY OF THE INVENTION

The present inventors have now discovered salts of N-{3-Chloro-4-[(3-fluorobenzyl)oxy]phenyl}-6-[5-({[2-(methanesulphonyl)ethyl]amino}methyl)-2-furyl]-4-quinazolinamine which may exhibit enhanced solubility in water and be suitable for bulk handling and formulation.

In a first aspect of the present invention, there is provided a salt of a compound of formula (I),

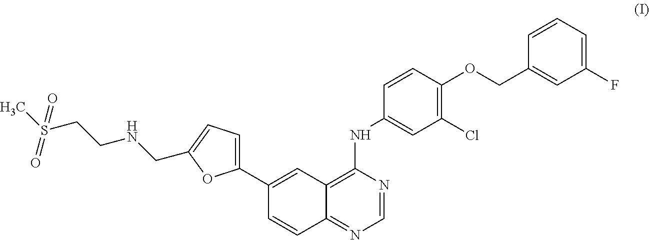

wherein said salt is an ethanesulfonate (esylate), methanesulfonate (mesylate), lactate, malate, maleate, benzoate, or citrate salt in non-solvated form, solvated form, or a mixture of non-solvated and solvated forms thereof.

In a second aspect of the present invention, there is provided a salt of a compound of formula (I), wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated and solvated forms thereof.

In a third aspect of the present invention, there is provided a pharmaceutical composition including a therapeutically effective amount of a salt of a compound of formula (I) wherein said salt is an ethanesulfonate (esylate), methanesulfonate (mesylate), lactate, malate, maleate, benzoate, or citrate salt in non-solvated form, solvated form, or a mixture of non-solvated and solvated forms thereof.

In a fourth aspect of the present invention, there is provided a pharmaceutical composition including a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, monocitrate salt in non-solvated form, solvated form, or a mixture of non-solvated and solvated forms thereof.

### BRIEF DESCRIPTION OF THE DRAWINGS

FIG. 1(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dimesylate dihydrate.

FIG. 1(b) depicts an infrared spectrum of N-{3-Chloro-4-[(3-fluorobenzyl) oxy]phenyl}-6-[5-({[2-(methanesulphonyl)ethyl]amino}methyl)-2-furyl]-4-quinazolinamine dimesylate dihydrate.

FIG. 1(b) depicts an Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimesylate dihydrate.

FIG. 2(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dimesylate monohydrate.

FIG. 2(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimesylate monohydrate.

FIG. 2(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimesylate monohydrate.

FIG. 3(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dimesylate.

FIG. 3(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimesylate.

FIG. 3(b) depicts an Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimesylate.

FIG. 4(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine monomesylate.

FIG. 4(b) depicts an infrared spectrum of N-{3-Chloro-4-[(3-fluorobenzyl) oxy]phenyl}-6-[5-({[2-(methanesulphonyl)ethyl]amino}methyl)-2-furyl]-4-quinazolinamine monomesylate.

FIG. 4(b) depicts an Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine monomesylate.

FIG. 5(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine diesylate.

FIG. 5(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine diesylate.

FIG. 5(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine diesylate.

FIG. 6(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine monoesylate.

FIG. 6(b) depicts an infrared spectrum of N-{3-Chloro-4-[(3-fluorobenzyl) oxy]phenyl}-6-[5-({[2-(methanesulphonyl)ethyl]amino}methyl)-2-furyl]-4-quinazolinamine monoesylate.

FIG. 6(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine monoesylate.

FIG. 7(a) depicts an X-ray powder pattern of N-{3-Chloro-4-[(3-fluorobenzyl) oxy]phenyl}-6-[5-({[2-(methanesulphonyl)ethyl]amino}methyl)-2-furyl]-4-quinazolinamine dimaleate.

FIG. 7(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimaleate.

FIG. 7(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimaleate.

FIG. 8(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dimaleate monohydrate.

FIG. 8(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimaleate monohydrate.

FIG. 8(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dimaleate monohydrate.

FIG. 9(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine di-L-lactate.

FIG. 9(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine di-L-lactate.

FIG. 9(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine di-L-lactate.

FIG. 10(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine mono-L-lactate.

FIG. 10(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine mono-L-lactate.

FIG. 10(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine mono-L-lactate.

FIG. 11(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine monocitrate.

FIG. 11(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine monocitrate.

FIG. 11(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine monocitrate.

FIG. 12(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dibenzoate.

FIG. 12(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dibenzoate.

FIG. 12(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dibenzoate.

FIG. 13(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine mono-L-malate.

FIG. 13(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine mono-L-malate.

FIG. 13(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine mono-L-malate.

FIG. 14(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine monosuccinate.

FIG. 14(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine monosuccinate.

FIG. 14(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine monosuccinate.

FIG. 15(a) depicts an X-ray powder diffraction pattern of N-{3-Chloro-4-[(3-fluorobenzyl)oxy]phenyl}-6-[5-({[2-(methanesulphonyl)ethyl]amino}methyl)-2-furyl]-4-quinazolinamine dibesylate.

FIG. 15(b) depicts an infrared spectrum of N-{3-Chloro-4-[(3-fluorobenzyl)oxy]phenyl}-6-[5-({[2-(methanesulphonyl)ethyl]amino}methyl)-2-furyl]-4-quinazolinamine dibesylate.

FIG. 15(b) depicts a Raman spectrum of N-{3-Chloro-4-[(3-fluorobenzyl)oxy]phenyl}-6-[5-({[2-(methanesulphonyl)ethyl]amino}methyl)-2-furyl]-4-quinazolinamine dibesylate.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “pharmaceutically acceptable salts” means those salts which are non-toxic and that are suitable for manufacturing and formulation as a pharmaceutical entity.

As used herein, the term “solvated” is understood to mean formation of a crystalline complex of variable stoichiometry comprising (in this invention), a compound of Formula (I) or a salt thereof and a solvent. Such solvents for the purpose of the invention may not interfere with the biological activity of the solute. Examples of suitable solvents include, but are not limited to, water, methanol, ethanol and acetic acid. Typically, the solvent used is a pharmaceutically acceptable solvent. Examples of suitable pharmaceutically acceptable solvents include water, ethanol and acetic acid. In one embodiment, the solvent used is water. In this case the solvate may be referred to as a hydrate.

As used herein, the term “non-solvated” is understood to mean the subject compound, i.e., the compound of formula (I) or salts thereof, have not formed a complex of variable stoichiometry with a solvent. A non-hydrated compound may be referred to as an anhydrate.

As used herein, the term “substantially the same X-ray powder diffraction pattern” is understood to mean that those X-ray powder diffraction patterns having diffraction peaks with 2 theta values within plus or minus 0.1° of the diffraction pattern referred to herein are within the scope of the referred to diffraction pattern. In a like manner, the term “at least substantially includes peaks of Table X” (where X is one of Tables 1-15) is understood to mean that those X-ray powder diffraction patterns having diffraction peaks with 2 theta values within plus or minus 0.1° of the subject Table are within the scope of the diffraction pattern referenced to the Table X. Also, in a like manner, the term “at least substantially includes the X-ray powder diffraction (XRPD) °2θ peaks Q1, Q2, Q3, . . . ” (where Q1, Q2, Q3, . . . represent specific listed peak two theta values) is understood to mean that those X-ray powder diffraction patterns having diffraction peaks with 2 theta values within plus or minus 0.1° of the subject listed peak two theta values are within the scope of the subject listed peak 2 theta values.

Also, as used herein, the term “substantially the same infrared spectrum” is understood to mean that those infrared spectrum (run according to the method described) having infrared peaks with cm⁻¹ values within plus or minus 2 cm⁻¹ of the spectrum referred to herein are within the scope of the referred to infrared spectrum.

Also, as used herein, the term “substantially the same Raman spectrum” is understood to mean that those Raman spectrum (run according to the method described) having Raman peaks with cm⁻¹ values within plus or minus 4 cm⁻¹ of the spectrum referred to herein are within the scope of the referred to Raman spectrum.

The present invention may include a salt of a compound of formula (I), wherein said salt is a fumarate, methanesulfonate, ethanesulfonate, lactate, benzenesulfonate (besylate), malate, maleate, benzoate, hydrobromide, tartrate, citrate, salicylate, succinate, or ethanedisulfonate (edisylate) salt in non-solvated form, solvated form, or a mixture of non-solvated or solvated forms thereof. In one embodiment, the salt is a pharmaceutically acceptable salt.

In another embodiment, the salt of a compound of formula (I) is an ethanesulfonate (esylate), methanesulfonate (mesylate), lactate, malate, maleate, benzoate, or citrate salt in non-solvated form, solvated form, or a mixture of non-solvated and solvated forms thereof. In one embodiment, the salt is a pharmaceutically acceptable salt.

It is also understood that the compound of formula (I) has more than one basic center and that where the salts of the present invention possess one or more acidic groups salts of different stoichiometry may form. Accordingly, also included in the present invention are the mono-salts having a 1:1 ratio of acid to compound of formula (I); di-salts having a 2:1 ratio of acid to compound of formula (I); and hemi-salts having a 1:2 ratio of acid to compound of formula (I).

Furthermore, it is also understood that since some of the acids used to form the salts of the present invention, such salts may have different stereoisomeric forms, for example the lactate salt may exist as L, D, or DL isomers or mixtures thereof; malate in L (−), D (+), or DL isomers or mixtures thereof; and tartrate may be L (+), D (−), or DL isomers or mixtures thereof.

The salts of the compounds of formula (I) include within their scope substantially pure non-solvated or solvated forms, as well as mixtures of non-solvated and solvated forms including hydrate and anhydrate forms. It is also understood, that such compounds include crystalline or amorphous forms and mixtures of crystalline and amorphous forms.

Accordingly, in another embodiment, the salt of the compound of formula (I) is a diesylate, monoesylate, dimesylate, monomesylate, di-L-lactate, mono-L-lactate, di-L-malate, mono-L-malate, dimaleate, dibenzoate, di-L-tartrate, mono-L-tartrate, or monocitrate salt in non-solvated form, solvated form, or a mixture of non-solvated and solvated forms thereof. In one embodiment, the salt is a pharmaceutically acceptable salt.

The compound of formula (I) has the chemical name N-{3-Chloro-4-[(3-fluorobenzyl)oxy]phenyl}-6-[5-({[2-(methanesulphonyl)ethyl]amino}methyl)-2-furyl]-4-quinazolinamine and is also known as GW572016X or lapatinib.

The intermediates and free base of the compound of Formula (I) may be prepared according to the procedures of International Patent Application No. PCT/EP99/00048, filed Jan. 8, 1999, and published as WO 99/35146 on Jul. 15, 1999, referred to above. Such references also teach preparation of various 4-quinazoliamine hydrochloride salts.

The intermediates, free-base, and ditosylate salts of the compound of Formula (I) may be prepared according to the procedures of International Patent Application No. PCT/US01/20706, filed Jun. 28, 2001, and published as WO 02/02552 on Jan. 10, 2002, or according to the procedures of International Patent Application No. PCT/US06/014447, filed Apr. 18, 2006, and published as WO 06/113649 on Oct. 26, 2006

Specific methods for the preparation of the specific salts of the present invention are provided in the Examples following.

In one embodiment, the salt of the compound of formula (I) is a methanesulfonate (mesylate) salt in non-solvated form, solvated form, or a mixture of non-solvated and solvated forms thereof.

In one embodiment, the salt of the compound of formula (I) is a dimesylate salt in non-solvated form, solvated form, or a mixture of non-solvated and solvated forms thereof. In one embodiment, the compound is a non-solvated form of the dimesylate salt of the compound of formula (I). In another embodiment, the compound is a solvated form of the dimesylate salt of the compound of formula (I). In one embodiment, the compound is a dihydrate form of the dimesylate salt of the compound of formula (I). In another embodiment, the compound is a monohydrate form of the dimesylate salt of the compound of formula (I). In another embodiment, the compound is an anhydrate form of the dimesylate salt of the compound of formula (I). In one embodiment, the compound is a mixture of non-solvated and solvated forms of the dimesylate salt of the compound of formula (I). In another embodiment, the compound is a mixture of hydrate and anhydrate forms of the dimesylate salt of the compound of formula (I).

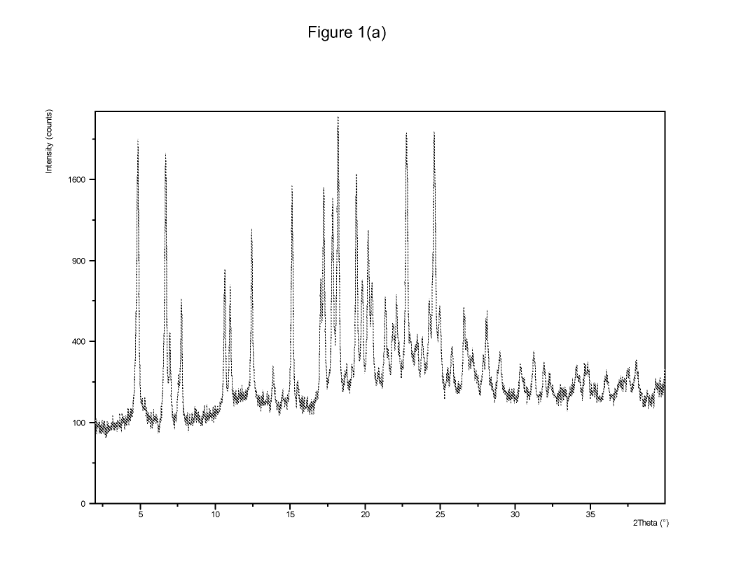

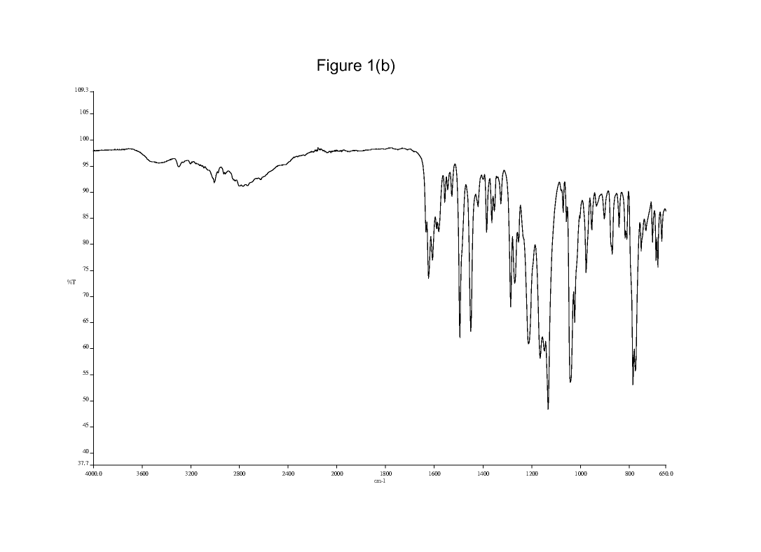

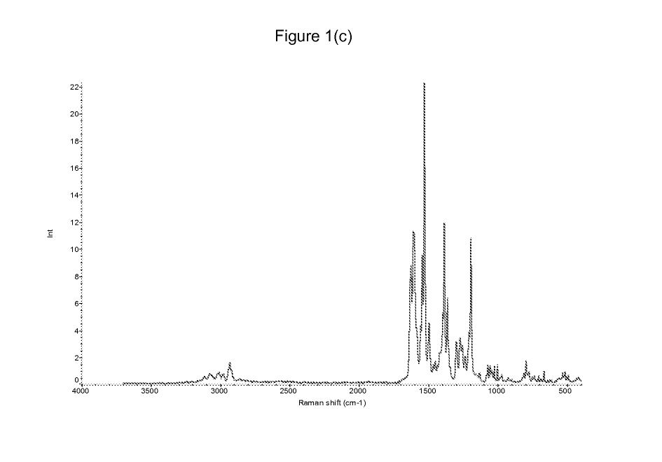

In a further embodiment, the compound is the dihydrate form of the dimesylate salt of the compound of formula (I) in crystalline form characterized by substantially the same X-ray powder diffraction pattern shown in FIG. 1(a). In another embodiment, the dihydrate form of the dimesylate salt of the compound of formula (I) in crystalline form is characterized by substantially the same infrared spectrum shown in FIG. 1(b). In another embodiment, the dihydrate form of the dimesylate salt of the compound of formula (I) in crystalline form is characterized by substantially the same Raman spectrum shown in FIG. 1(b). In another embodiment, the dihydrate of the dimesylate salt of the compound of formula (I) in crystalline form is characterized by an X-ray powder diffraction pattern which at least substantially includes the peaks of Table I.

TABLE I

| Pos.[°2Th.] | d-spacing[Å] |
|---|---|
| 4.8 | 18.2 |
| 6.7 | 13.2 |
| 7.0 | 12.7 |
| 7.8 | 11.4 |
| 10.6 | 8.3 |
| 11.0 | 8.0 |
| 12.4 | 7.1 |
| 13.9 | 6.4 |
| 15.1 | 5.9 |
| 17.0 | 5.2 |
| 17.2 | 5.1 |
| 17.8 | 5.0 |
| 18.2 | 4.9 |
| 19.4 | 4.6 |
| 19.8 | 4.5 |
| 20.2 | 4.4 |
| 20.4 | 4.3 |
| 21.3 | 4.2 |
| 22.1 | 4.0 |
| 22.8 | 3.9 |
| 24.3 | 3.7 |
| 24.6 | 3.6 |
| 25.0 | 3.6 |
| 26.6 | 3.4 |

* Based on Cu Kα radiation.

In another embodiment, the dihydrate of the dimesylate salt of the compound of formula (I) in crystalline form is characterized by an X-ray powder diffraction pattern which at least substantially includes the X-ray powder diffraction (XRPD) °2θ peaks 4.8, 6.7, 12.4, 15.1 and 22.8.

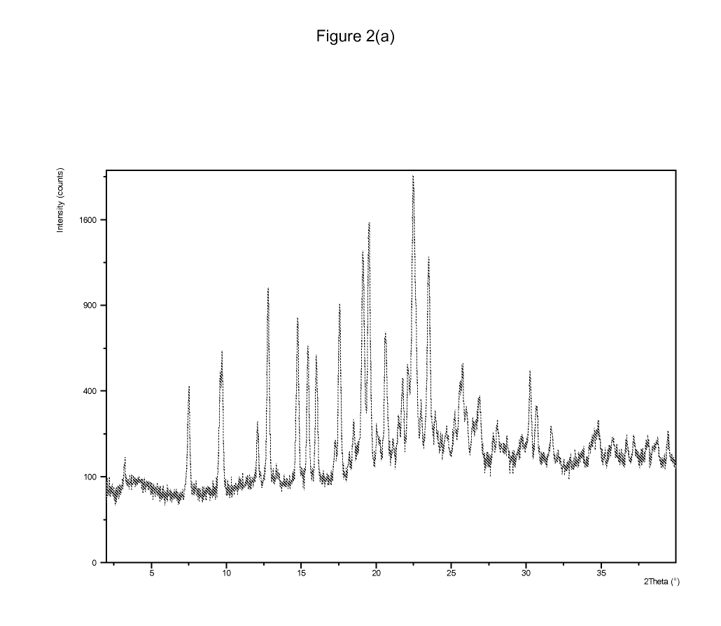

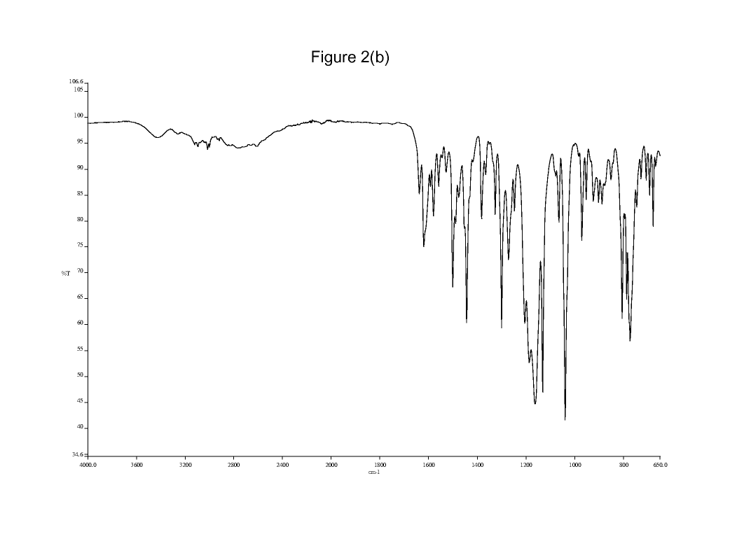

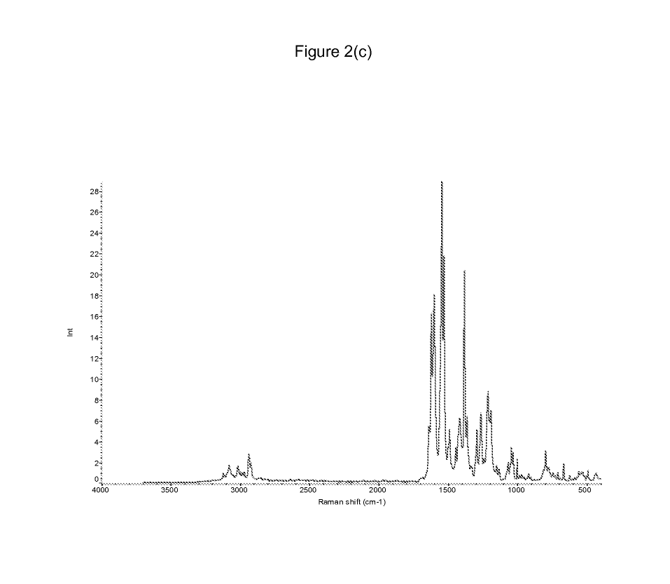

In a further embodiment, the compound is the monohydrate of the dimesylate salt of the compound of formula (I) in crystalline form characterized by substantially the same X-ray powder diffraction pattern shown in FIG. 2(a). In another embodiment, the monohydrate form of the dimesylate salt of the compound of formula (I) in crystalline form is characterized by substantially the same infrared spectrum shown in FIG. 2(b). In another embodiment, the monohydrate form of the dimesylate salt of the compound of formula (I) in crystalline form is characterized by substantially the same Raman spectrum shown in FIG. 2(b). In another embodiment, the monohydrate dimesylate salt of the compound of formula (I) in crystalline form is characterized by an X-ray powder diffraction pattern which substantially includes the peaks of Table II.

TABLE II

| Pos.[°2Th.] | d-spacing[Å] |
|---|---|
| 3.2 | 27.2 |
| 7.5 | 11.8 |
| 9.6 | 9.2 |
| 9.7 | 9.1 |
| 12.1 | 7.3 |
| 12.8 | 6.9 |
| 14.8 | 6.0 |
| 15.4 | 5.7 |
| 16.0 | 5.5 |
| 17.6 | 5.0 |
| 18.5 | 4.8 |
| 19.1 | 4.6 |
| 19.5 | 4.6 |
| 20.6 | 4.3 |
| 21.8 | 4.1 |
| 22.1 | 4.0 |
| 22.5 | 4.0 |
| 23.0 | 3.9 |
| 23.5 | 3.8 |
| 23.9 | 3.7 |
| 25.6 | 3.5 |
| 25.8 | 3.5 |
| 26.0 | 3.4 |
| 26.9 | 3.3 |
| 30.2 | 3.0 |
| 30.7 | 2.9 |

* Based on Cu Kα radiation.

In another embodiment, the monohydrate of the dimesylate salt of the compound of formula (I) in crystalline form is characterized by an X-ray powder diffraction pattern which at least substantially includes the XRPD °2θ peaks 7.5, 12.8, 14.8, 15.4 and 16.0.

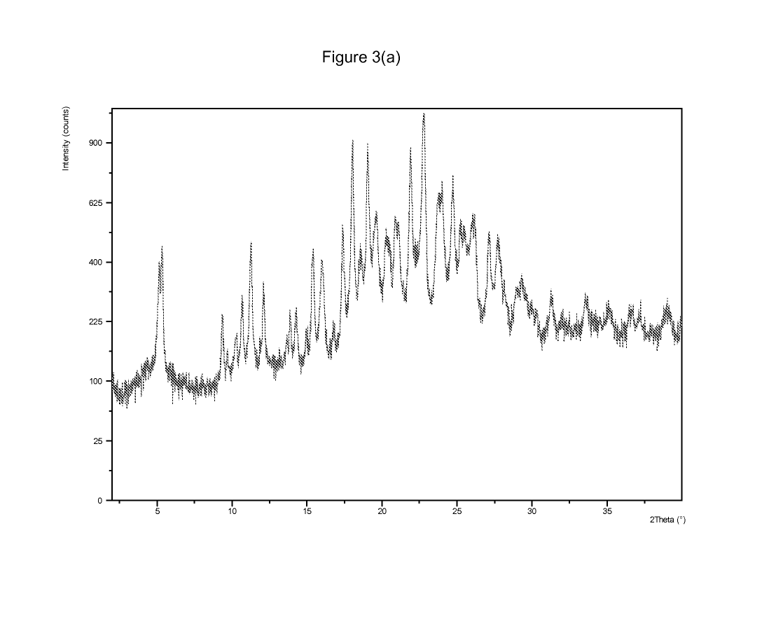

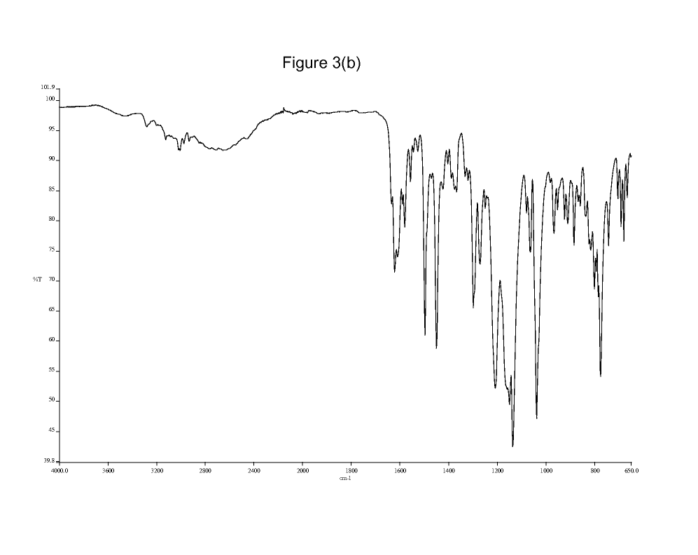

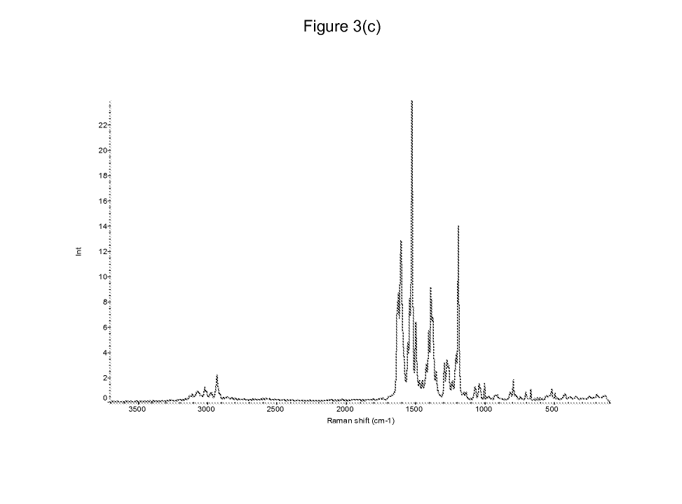

In another embodiment, the compound is a dimesylate salt of the compound of formula (I) in crystalline form characterized by substantially the same X-ray powder diffraction pattern shown in FIG. 3(a). In another embodiment, the dimesylate salt of the compound of formula (I) in crystalline form is characterized by substantially the same infrared spectrum shown in FIG. 3(b). In another embodiment, the dimesylate salt of the compound of formula (I) in crystalline form is characterized by substantially the same Raman spectrum shown in FIG. 3(b). In another embodiment, the dimesylate salt of the compound of formula (I) in crystalline form is characterized by a X-ray powder diffraction pattern which substantially includes the peaks of Table III.

TABLE III

| Pos.[°2Th.] | d-spacing[Å] |
|---|---|
| 5.2 | 17.1 |
| 5.4 | 16.5 |
| 9.4 | 9.4 |
| 10.3 | 8.6 |
| 10.6 | 8.3 |
| 11.3 | 7.8 |
| 12.1 | 7.3 |
| 13.8 | 6.4 |
| 14.3 | 6.2 |
| 15.0 | 5.9 |
| 15.4 | 5.7 |
| 16.0 | 5.5 |
| 16.8 | 5.3 |
| 17.4 | 5.1 |
| 18.0 | 4.9 |
| 18.6 | 4.8 |
| 19.0 | 4.7 |
| 19.6 | 4.5 |
| 20.2 | 4.4 |
| 20.9 | 4.3 |
| 21.1 | 4.2 |
| 21.9 | 4.1 |
| 22.8 | 3.9 |
| 23.7 | 3.8 |
| 24.0 | 3.7 |
| 24.7 | 3.6 |
| 25.2 | 3.5 |
| 26.2 | 3.4 |
| 27.1 | 3.3 |
| 27.7 | 3.2 |

* Based on Cu Kα radiation.

In another embodiment, the dimesylate salt of the compound of formula (I) in crystalline form is characterized by an X-ray powder diffraction pattern which at least substantially includes the XRPD °2θ peaks 5.4, 11.3, 15.4, 16.0 and 18.0.

In one embodiment, the salt of the compound of formula (I) is a monomesylate salt in non-solvated form, solvated form, or a mixture of non-solvated and solvated forms thereof. In one embodiment, the compound is a non-solvated form of the monomesylate salt of the compound of formula (I). In another embodiment, the compound is a solvated form of the monomesylate salt of the compound of formula (I). In one embodiment, the compound is a hydrated form of the monomesylate salt of the compound of formula (I). In another embodiment, the compound is an anhydrate form of the monomesylate salt of the compound of formula (I). In one embodiment, the compound is a mixture of non-solvated and solvated forms of the monomesylate salt of the compound of formula (I). In another embodiment, the compound is a mixture of hydrate and anhydrate forms of the monomesylate salt of the compound of formula (I).

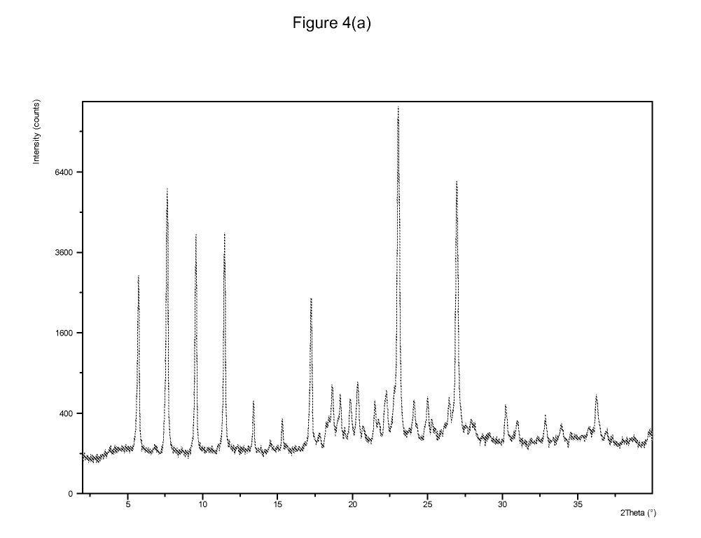

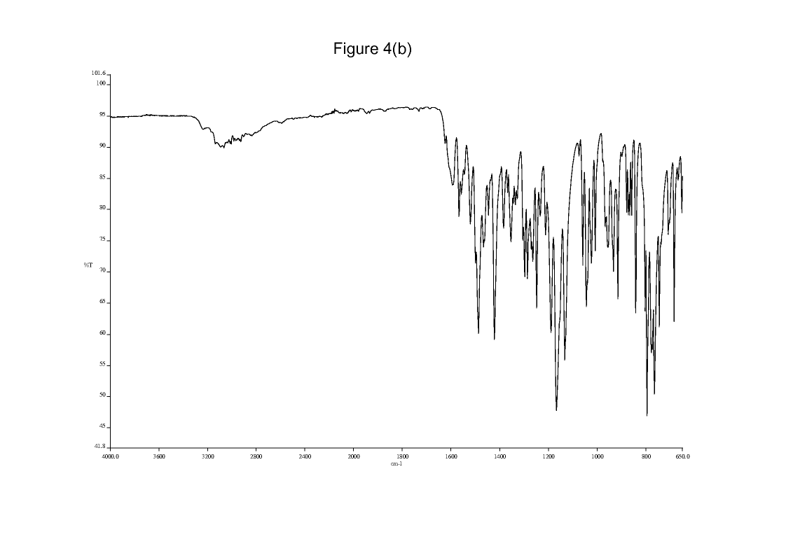

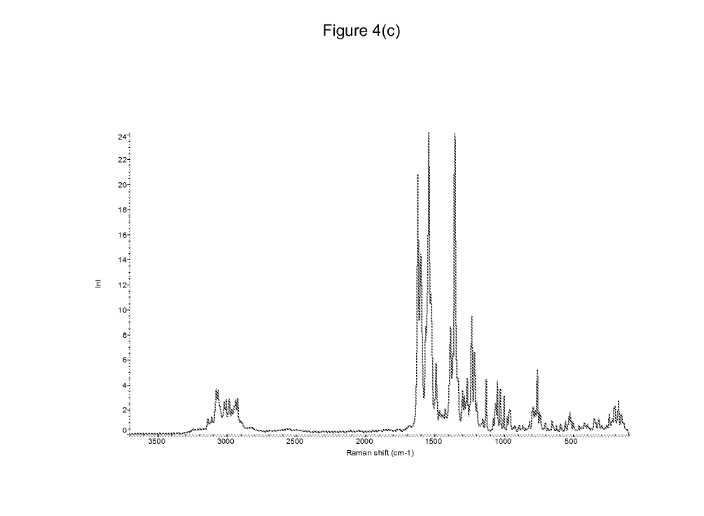

In another embodiment, the compound is a monomesylate salt of the compound of formula (I) in crystalline form characterized by substantially the same X-ray powder diffraction pattern shown in FIG. 4(a). In another embodiment, the monomesylate salt of the compound of formula (I) in crystalline form is characterized by substantially the same infrared spectrum shown in FIG. 4(b). In another embodiment, the monomesylate salt of the compound of formula (I) in crystalline form is characterized by substantially the same Raman spectrum shown in FIG. 4(b). In another embodiment, the monomesylate salt of the compound of formula (I) in crystalline form is characterized by a X-ray powder diffraction pattern which substantially includes the peaks of Table IV.

TABLE IV

| | d- |
|---|---|
| Pos. [°2Th.] | spacing [Å] |
| 5.7 | 15.4 |
| 7.6 | 11.6 |
| 9.6 | 9.3 |
| 11.5 | 7.7 |
| 13.4 | 6.6 |
| 15.3 | 5.8 |
| 17.3 | 5.1 |
| 18.7 | 4.8 |
| 19.2 | 4.6 |
| 19.8 | 4.5 |
| 20.3 | 4.4 |
| 21.5 | 4.1 |
| 22.3 | 4.0 |
| 23.1 | 3.9 |
| 24.1 | 3.7 |
| 25.0 | 3.6 |
| 26.4 | 3.4 |
| 27.0 | 3.3 |
| 30.2 | 3.0 |
| 36.2 | 2.5 |

Based on Cu Kα radiation.

In another embodiment, the monomesylate salt of the compound of formula (I) in crystalline form is characterized by an X-ray powder diffraction pattern which at least substantially includes the XRPD °2θ peaks 5.7, 7.6, 9.6, 11.5 and 17.3.

In one embodiment, the salt of the compound of formula (I) is a ethanesulfonate (esylate) salt in non-solvated form, solvated form, or a mixture of non-solvated and solvated forms thereof.

In one embodiment, the salt of the compound of formula (I) is a diesylate salt in non-solvated form, solvated form, or a mixture of non-solvated or solvated forms thereof. In one embodiment, the compound is a non-solvated form of the diesylate salt of the compound of formula (I). In another embodiment, the compound is a solvated form of the diesylate salt of the compound of formula (I). In one embodiment, the compound is a hydrated form of the diesylate salt of the compound of formula (I). In another embodiment, the compound is an anhydrate form of the diesylate salt of the compound of formula (I). In another embodiment, the compound is a mixture of non-solvated and solvated forms of the diesylate salt of the compound of formula (I). In another embodiment, the compound is a mixture of hydrate and anhydrate forms of the diesylate salt of the compound of formula (I).

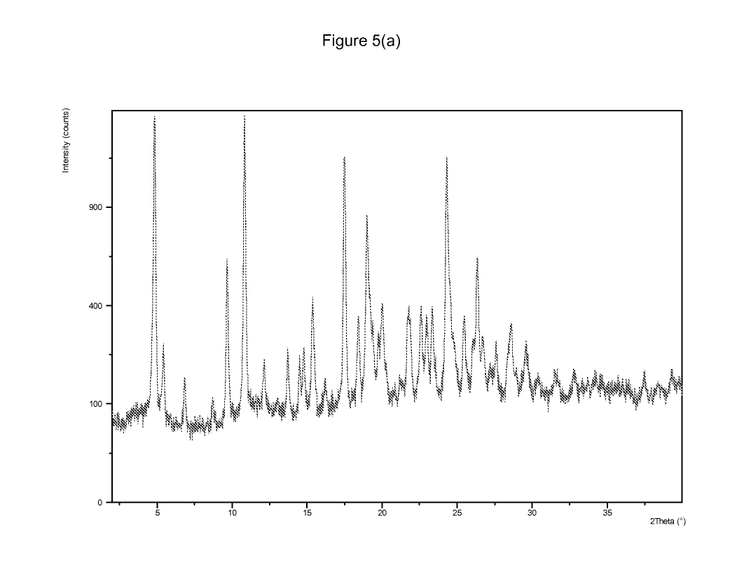

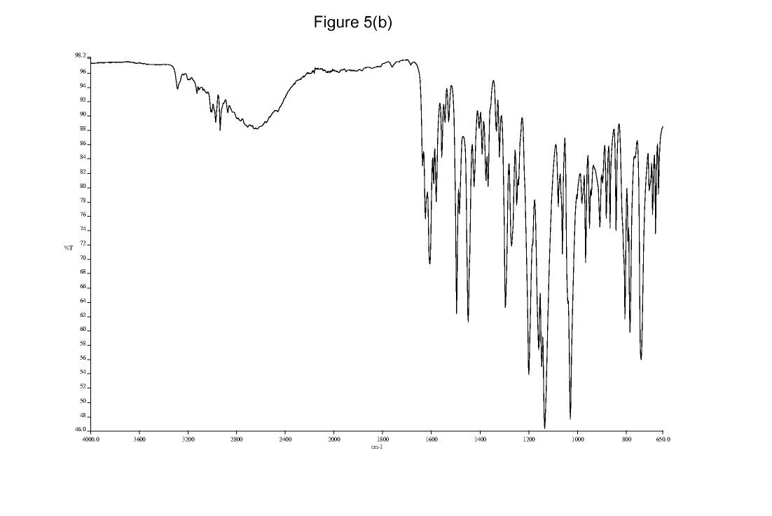

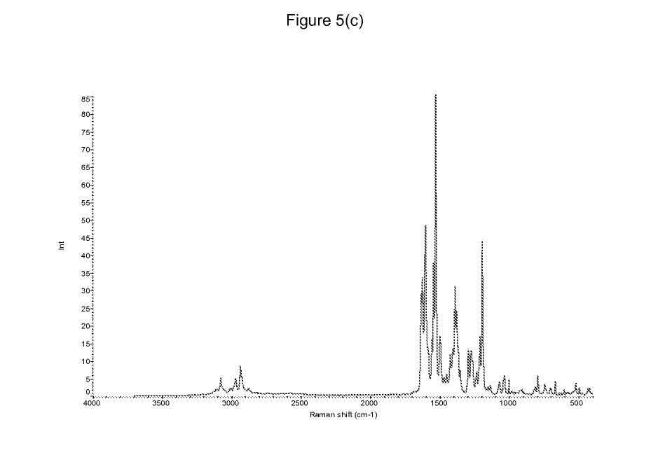

In another embodiment, the compound is a diesylate salt of the compound of formula (I) in crystalline form characterized by substantially the same X-ray powder diffraction pattern shown in FIG. 5(a). In another embodiment, the compound is a diesylate salt of the compound of formula (I) in crystalline form characterized by substantially the same infrared spectrum shown in FIG. 5(b). In another embodiment, the compound is a diesylate salt of the compound of formula (I) in crystalline form characterized by substantially the same Raman spectrum shown in FIG. 5(b). In another embodiment, the diesylate salt of the compound of formula (I) in crystalline form is characterized by an X-ray powder diffraction pattern which substantially includes the peaks of Table V.

TABLE V

| | d- |
|---|---|
| Pos. [°2Th.] | spacing [Å] |
| 4.8 | 18.3 |
| 5.4 | 16.3 |
| 6.9 | 12.9 |
| 8.7 | 10.2 |
| 9.7 | 9.1 |
| 10.8 | 8.2 |
| 12.1 | 7.3 |
| 13.7 | 6.5 |
| 14.5 | 6.1 |
| 14.8 | 6.0 |
| 15.4 | 5.8 |
| 17.5 | 5.1 |
| 18.4 | 4.8 |
| 19.0 | 4.7 |
| 19.8 | 4.5 |
| 20.0 | 4.4 |
| 21.7 | 4.1 |
| 22.6 | 3.9 |
| 22.9 | 3.9 |
| 23.3 | 3.8 |
| 24.3 | 3.7 |
| 25.5 | 3.5 |
| 26.3 | 3.4 |
| 28.6 | 3.1 |

* Based on Cu Kα radiation.

In another embodiment, the diesylate salt of the compound of formula (I) in crystalline form is characterized by an X-ray powder diffraction pattern which at least substantially includes the XRPD °2θ peaks 4.8, 9.7, 10.8, 17.5 and 24.3.

In another embodiment, the salt of the compound of formula (I) is a monoesylate salt in non-solvated form, solvated form, or a mixture of non-solvated or solvated forms thereof. In one embodiment, the compound is a non-solvated form of the monoesylate salt of the compound of formula (I). In another embodiment, the compound is a solvated form of the monoesylate salt of the compound of formula (I). In one embodiment, the compound is a hydrated form of the monoesylate salt of the compound of formula (I). In another embodiment, the compound is an anhydrate form of the monoesylate salt of the compound of formula (I). In another embodiment, the compound is a mixture of non-solvated and solvated forms of the monoesylate salt of the compound of formula (I). In another embodiment, the compound is a mixture of hydrate and anhydrate forms of the monoesylate salt of the compound of formula (I).

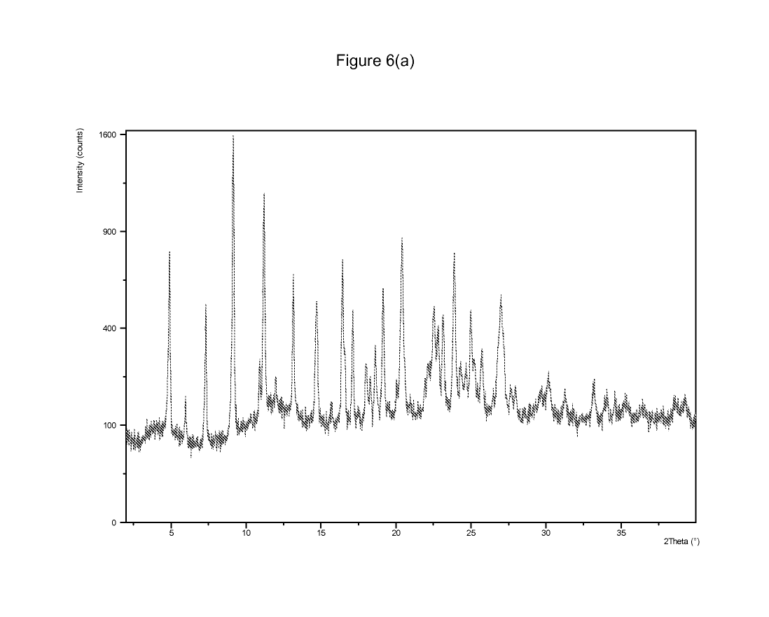

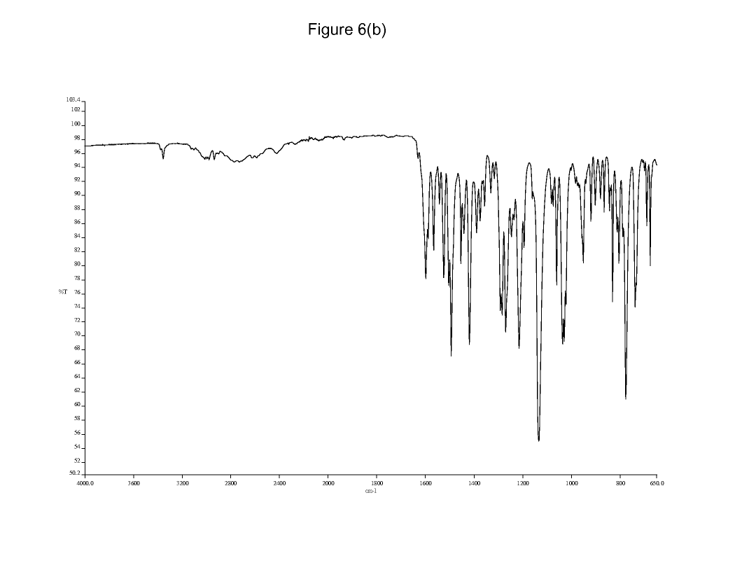

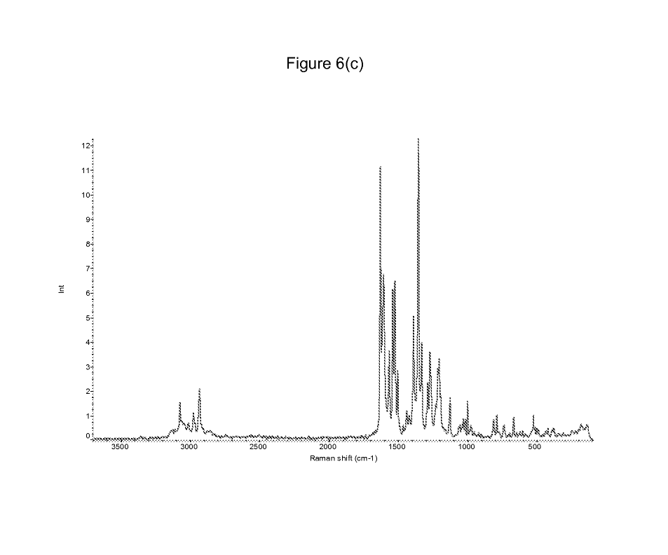

In another embodiment, the compound is a monoesylate salt of the compound of formula (I) in crystalline form characterized by substantially the same X-ray powder diffraction pattern shown in FIG. 6(a). In another embodiment, the compound is a monoesylate salt of the compound of formula (I) in crystalline form characterized by substantially the same infrared spectrum shown in FIG. 6(b). In another embodiment, the compound is a monoesylate salt of the compound of formula (I) in crystalline form characterized by substantially the same Raman spectrum shown in FIG. 6(b). In another embodiment, the monoesylate salt of the compound of formula (I) in crystalline form is characterized by an X-ray powder diffraction pattern which substantially includes the peaks of Table VI.

TABLE VI

| | d- |
|---|---|
| Pos. [°2Th.] | spacing [Å] |
| 4.9 | 18.0 |
| 6.0 | 14.8 |
| 7.3 | 12.1 |
| 9.1 | 9.7 |
| 10.9 | 8.1 |
| 11.2 | 7.9 |
| 12.0 | 7.4 |
| 13.2 | 6.7 |
| 14.7 | 6.0 |
| 15.7 | 5.6 |
| 16.4 | 5.4 |
| 17.1 | 5.2 |
| 18.0 | 4.9 |
| 18.6 | 4.8 |
| 19.1 | 4.6 |
| 20.4 | 4.4 |
| 22.5 | 3.9 |
| 22.8 | 3.9 |
| 23.1 | 3.8 |
| 23.9 | 3.7 |
| 25.0 | 3.6 |
| 25.7 | 3.5 |
| 27.0 | 3.3 |

* Based on Cu Kα radiation.

In another embodiment, the monoesylate salt of the compound of formula (I) in crystalline form is characterized by an X-ray powder diffraction pattern which at least substantially includes the XRPD °2θ peaks 4.9, 7.3, 9.1, 11.2, and 16.4.

In one embodiment, the salt of the compound of formula (I) is a maleate salt in non-solvated form, solvated form, or a mixture of non-solvated or solvated forms thereof.

In one embodiment, the salt of the compound of formula (I) is a dimaleate salt in non-solvated form, solvated form, or a mixture of non-solvated or solvated forms thereof. In one embodiment, the compound is a non-solvated form of the dimaleate salt of the compound of formula (I). In another embodiment, the compound is a solvated form of the dimaleate salt of the compound of formula (I). In one embodiment, the compound is a hydrated form of the dimaleate salt of the compound of formula (I). In another embodiment, the compound is an anhydrate form of the dimaleate salt of the compound of formula (I). In another embodiment, the compound is a mixture of non-solvated and solvated forms of the dimaleate salt of the compound of formula (I). In another embodiment, the compound is a mixture of hydrate and anhydrate forms of the dimaleate salt of the compound of formula (I).

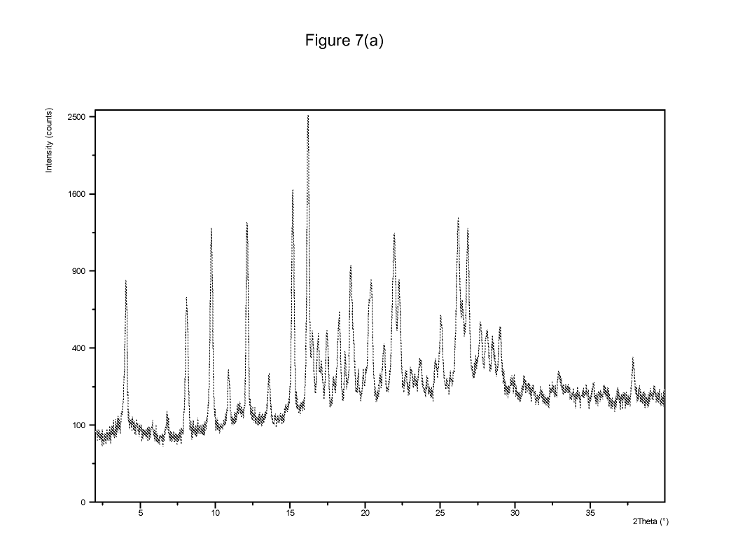

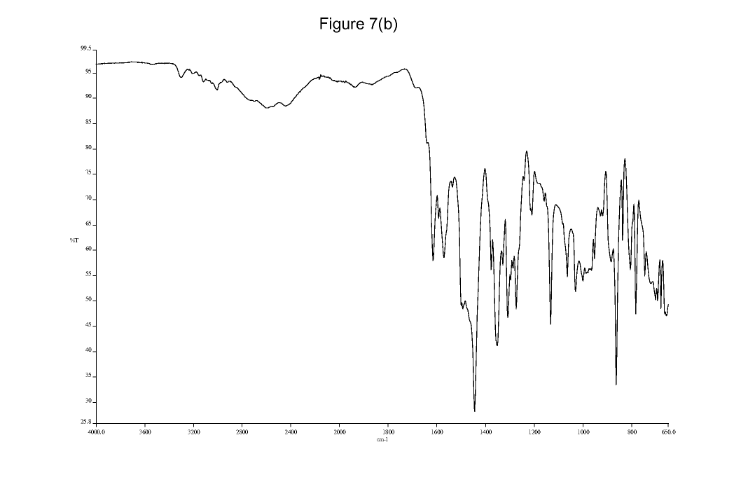

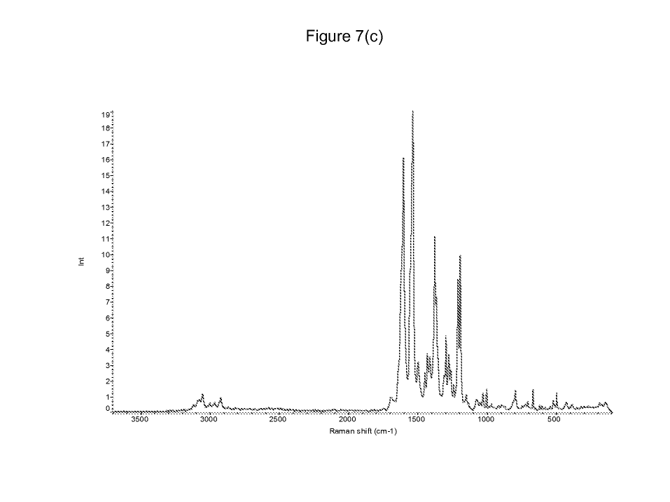

In another embodiment, the compound is an anhydrate of the dimaleate salt of the compound of formula (I) in crystalline form characterized by substantially the same X-ray powder diffraction pattern shown in FIG. 7(a). In another embodiment, the compound is an anhydrate of the dimaleate salt of the compound of formula (I) in crystalline form characterized by substantially the same infrared spectrum shown in FIG. 7(b). In another embodiment, the compound is an anhydrate of the dimaleate salt of the compound of formula (I) in crystalline form characterized by substantially the same Raman spectrum shown in FIG. 7(b). In another embodiment, the dimaleate salt of the compound of formula (I) in crystalline form is characterized by an X-ray powder diffraction pattern which substantially includes the peaks of Table VII.

TABLE VII

| | d- |
|---|---|
| Pos. [°2Th.] | spacing [Å] |
| 4.1 | 21.8 |
| 6.8 | 12.9 |
| 8.1 | 11.0 |
| 9.7 | 9.1 |
| 10.9 | 8.1 |
| 12.1 | 7.3 |
| 13.6 | 6.5 |
| 15.2 | 5.8 |
| 16.2 | 5.5 |
| 16.5 | 5.4 |
| 16.9 | 5.2 |
| 17.5 | 5.1 |
| 18.3 | 4.9 |
| 19.0 | 4.7 |
| 20.4 | 4.3 |
| 21.3 | 4.2 |
| 21.9 | 4.1 |
| 22.2 | 4.0 |
| 25.0 | 3.6 |
| 26.2 | 3.4 |
| 26.9 | 3.3 |
| 27.7 | 3.2 |
| 28.1 | 3.2 |
| 28.5 | 3.1 |
| 29.0 | 3.1 |

*Based on Cu Kα radiation

In another embodiment, the anhydrite form of the dimaleate salt of the compound of formula (I) in crystalline form is characterized by an X-ray powder diffraction pattern which at least substantially includes the XRPD °2θ peaks 4.1, 9.7, 12.1, 15.2 and 16.2.

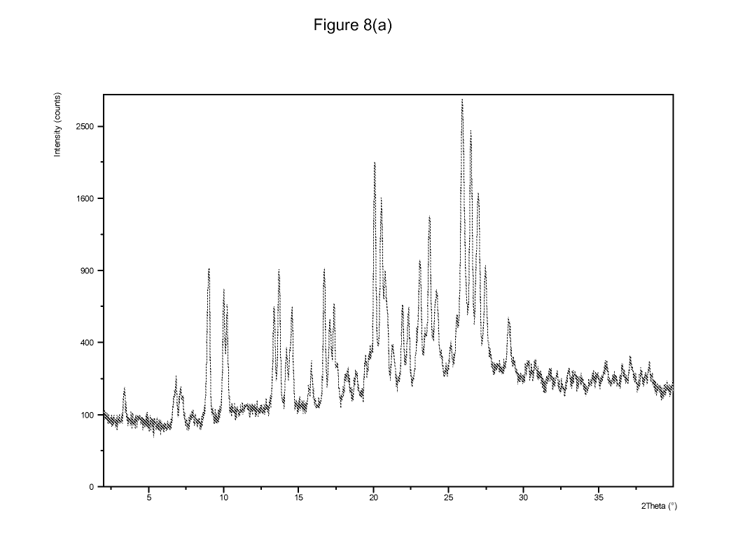

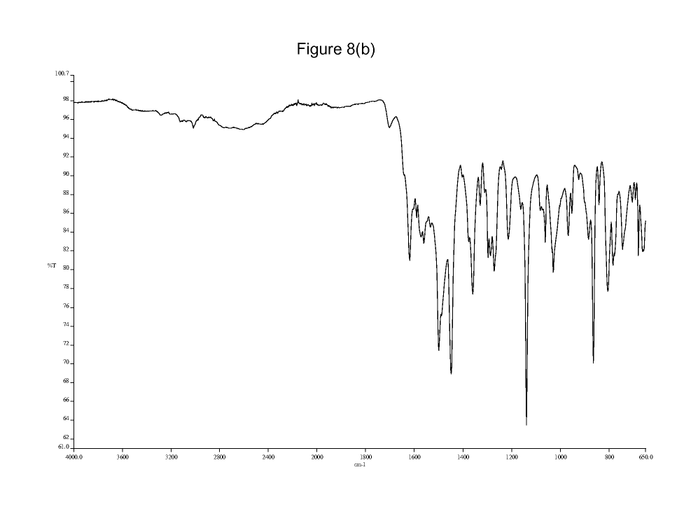

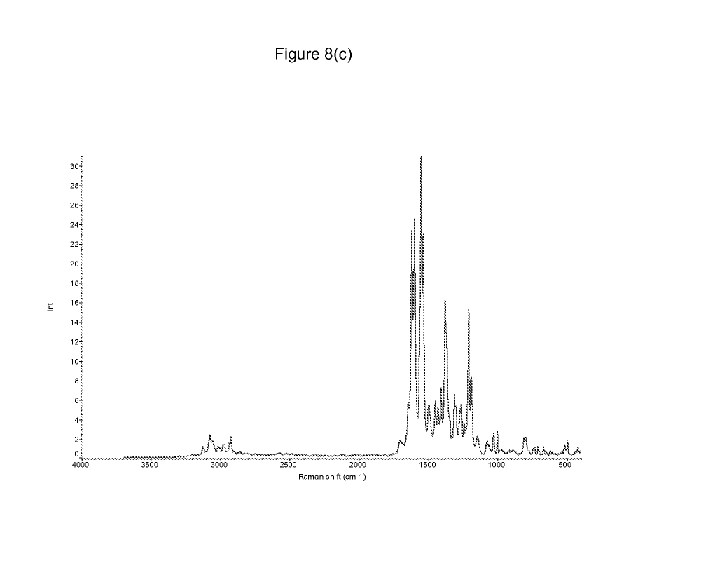

In another embodiment, the compound is a dimaleate monohydrate salt of the compound of formula (I) in crystalline form characterized by substantially the same X-ray powder diffraction pattern shown in FIG. 8(a). In another embodiment, the compound is a dimaleate monhydrate salt of the compound of formula (I) in crystalline form characterized by substantially the same infrared spectrum shown in FIG. 8(b). In another embodiment, the compound is a dimaleate monhydrate salt of the compound of formula (I) in crystalline form characterized by substantially the same Raman spectrum shown in FIG. 8(b). In another embodiment, the dimaleate monohydrate salt of the compound of formula (I) in crystalline form is characterized by an X-ray powder diffraction pattern which substantially includes the peaks of Table VIII.

TABLE VIII

| | d- |
|---|---|
| Pos. [°2Th.] | spacing [Å] |
| 3.3 | 26.5 |
| 6.8 | 12.9 |
| 7.1 | 12.4 |
| 7.3 | 12.1 |
| 9.0 | 9.8 |
| 10.0 | 8.8 |
| 10.2 | 8.6 |
| 13.4 | 6.6 |
| 13.7 | 6.5 |
| 14.2 | 6.2 |
| 14.6 | 6.1 |
| 15.7 | 5.7 |
| 15.9 | 5.6 |
| 16.7 | 5.3 |
| 17.1 | 5.2 |
| 17.4 | 5.1 |
| 20.1 | 4.4 |
| 20.5 | 4.3 |
| 20.8 | 4.3 |
| 21.0 | 4.2 |
| 21.9 | 4.1 |
| 22.3 | 4.0 |
| 23.1 | 3.8 |
| 23.8 | 3.7 |
| 24.2 | 3.7 |
| 25.5 | 3.5 |
| 25.9 | 3.4 |
| 26.5 | 3.4 |
| 27.0 | 3.3 |
| 27.5 | 3.2 |

* Based on Cu Kα radiation.

In another embodiment, the dimaleate monohydrate salt of the compound of formula (I) in crystalline form is characterized by an X-ray powder diffraction pattern which at least substantially includes the XRPD °2θ peaks 9.0, 10.0, 10.2, 13.4 and 14.6.

In one embodiment, the salt of the compound of formula (I) is a lactate salt in non-solvated form, solvated form, or a mixture of non-solvated or solvated forms thereof.

In one embodiment, the salt of the compound of formula (I) is a dilactate salt in non-solvated form, solvated form, or a mixture of non-solvated or solvated forms thereof. In one embodiment, the compound is a non-solvated form of the dilactate salt of the compound of formula (I). In another embodiment, the compound is a solvated form of the dilactate salt of the compound of formula (I). In one embodiment, the compound is a hydrated form of the dilactate salt of the compound of formula (I). In another embodiment, the compound is an anhydrate form of the dilactate salt of the compound of formula (I). In another embodiment, the compound is a mixture of non-solvated and solvated forms of the dilactate salt of the compound of formula (I). In another embodiment, the compound is a mixture of hydrate and anhydrate forms of the dilactate salt of the compound of formula (I).

In one embodiment, the salt of the compound of formula (I) is a di-L-lactate salt in non-solvated form, solvated form, or a mixture of non-solvated or solvated forms thereof. In one embodiment, the compound is a non-solvated form of the di-L-lactate salt of the compound of formula (I). In another embodiment, the compound is a solvated form of the di-L-lactate salt of the compound of formula (I). In one embodiment, the compound is a hydrated form of the di-L-lactate salt of the compound of formula (I). In another embodiment, the compound is an anhydrate form of the di-L-lactate salt of the compound of formula (I). In another embodiment, the compound is a mixture of non-solvated and solvated forms of the di-L-lactate salt of the compound of formula (I). In another embodiment, the compound is a mixture of hydrate and anhydrate forms of the di-L-lactate salt of the compound of formula (I).

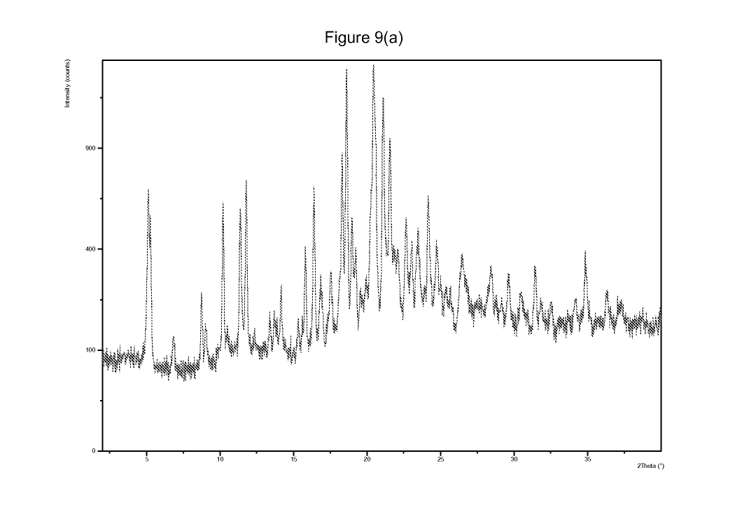

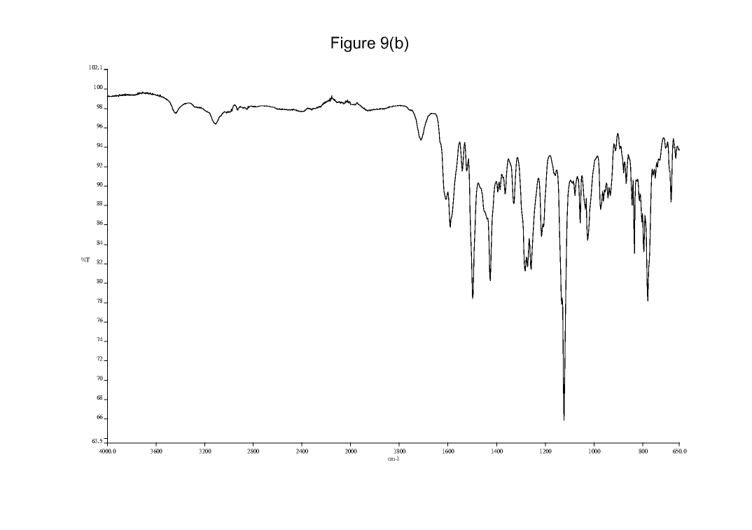

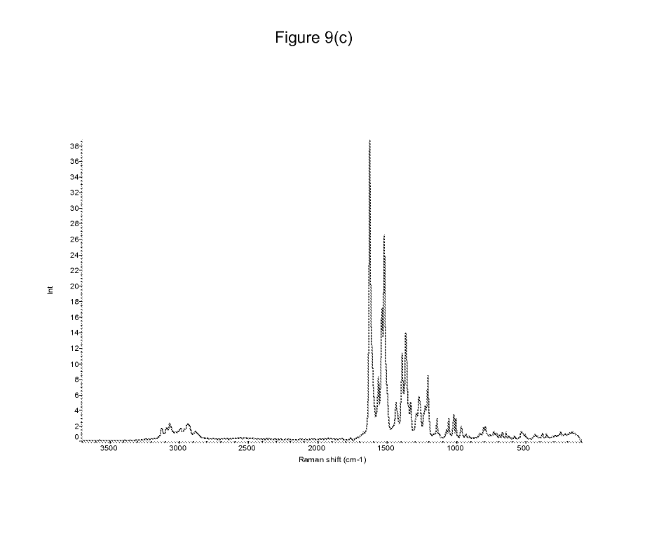

In another embodiment, the compound is a di-L-lactate salt of the compound of formula (I) in crystalline form characterized by substantially the same X-ray powder diffraction pattern shown in FIG. 9(a). In another embodiment, the compound is a di-L-lactate salt of the compound of formula (I) in crystalline form characterized by substantially the same infrared spectrum shown in FIG. 9(b). In another embodiment, the compound is a di-L-lactate salt of the compound of formula (I) in crystalline form characterized by substantially the same Raman spectrum shown in FIG. 9(b). In another embodiment, the di-L-lactate salt of the compound of formula (I) in crystalline form is characterized by an X-ray powder diffraction pattern which substantially includes the peaks of Table IX.

TABLE IX

| | d- |
|---|---|
| Pos. [°2Th.] | spacing [Å] |
| 5.1 | 17.3 |
| 5.3 | 16.8 |
| 6.8 | 12.9 |
| 8.7 | 10.1 |
| 9.0 | 9.8 |
| 10.2 | 8.7 |
| 11.4 | 7.8 |
| 11.8 | 7.5 |
| 12.4 | 7.2 |
| 13.4 | 6.6 |
| 13.7 | 6.5 |
| 14.2 | 6.3 |
| 15.3 | 5.8 |
| 15.8 | 5.6 |
| 16.4 | 5.4 |
| 16.8 | 5.3 |
| 17.6 | 5.1 |
| 18.3 | 4.8 |
| 18.6 | 4.8 |
| 19.0 | 4.7 |
| 19.2 | 4.6 |
| 19.5 | 4.5 |
| 20.4 | 4.3 |
| 20.6 | 4.3 |
| 21.1 | 4.2 |
| 21.6 | 4.1 |
| 22.1 | 4.0 |
| 22.7 | 3.9 |
| 23.1 | 3.9 |
| 23.5 | 3.8 |
| 24.1 | 3.7 |
| 24.8 | 3.6 |
| 26.5 | 3.4 |
| 34.8 | 2.6 |

* Based on Cu Kα radiation.

In another embodiment, the di-L-lactate salt of the compound of formula (I) in crystalline form is characterized by an X-ray powder diffraction pattern which at least substantially includes the XRPD °2θ peaks 5.1, 10.2, 11.4, 11.8 and 21.1.

In one embodiment, the salt of the compound of formula (I) is a monolactate salt in non-solvated form, solvated form, or a mixture of non-solvated or solvated forms thereof. In one embodiment, the compound is a non-solvated form of the monolactate salt of the compound of formula (I). In another embodiment, the compound is a solvated form of the monolactate salt of the compound of formula (I). In one embodiment, the compound is a hydrated form of the monolactate salt of the compound of formula (I). In another embodiment, the compound is an anhydrate form of the monolactate salt of the compound of formula (I). In another embodiment, the compound is a mixture of non-solvated and solvated forms of the monolactate salt of the compound of formula (I). In another embodiment, the compound is a mixture of hydrate and anhydrate forms of the monolactate salt of the compound of formula (I).

In one embodiment, the salt of the compound of formula (I) is a mono-L-lactate salt in non-solvated form, solvated form, or a mixture of non-solvated or solvated forms thereof. In one embodiment, the compound is a non-solvated form of the mono-L-lactate salt of the compound of formula (I). In another embodiment, the compound is a solvated form of the mono-l-lactate salt of the compound of formula (I). In one embodiment, the compound is a hydrated form of the mono-L-lactate salt of the compound of formula (I). In another embodiment, the compound is an anhydrate form of the mono-L-lactate salt of the compound of formula (I). In another embodiment, the compound is a mixture of non-solvated and solvated forms of the mono-L-lactate salt of the compound of formula (I). In another embodiment, the compound is a mixture of hydrate and anhydrate forms of the mono-L-lactate salt of the compound of formula (I).

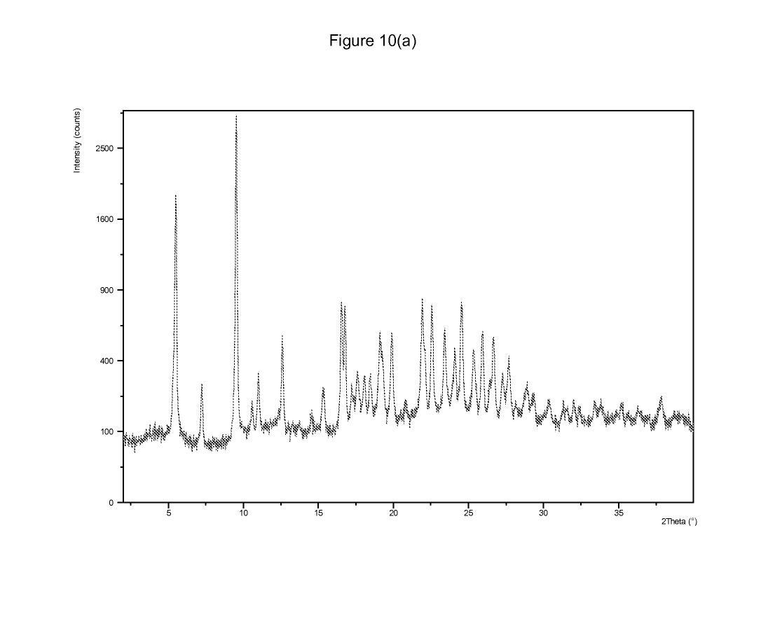

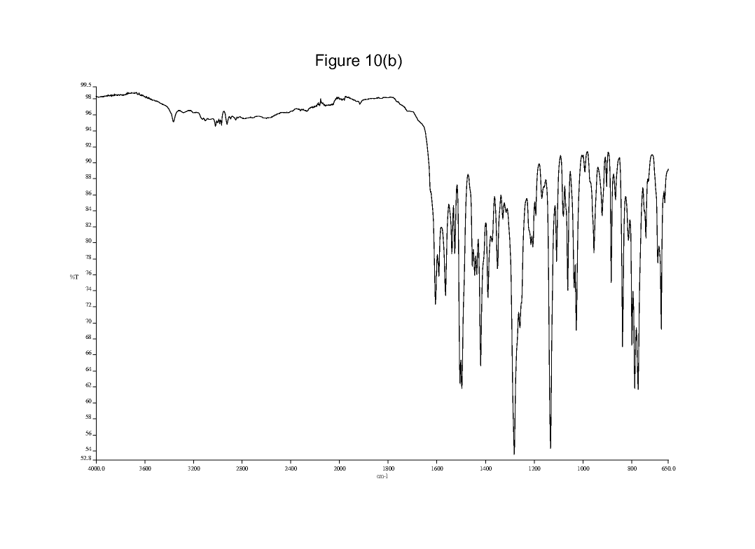

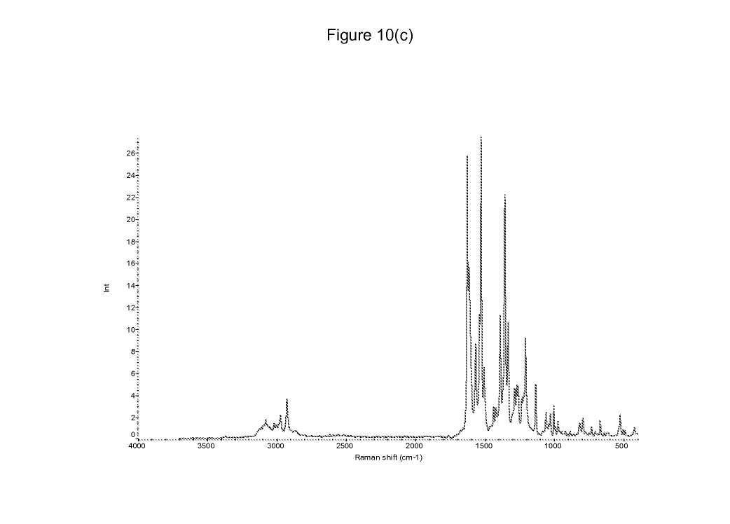

In another embodiment, the compound is a mono-L-lactate salt of the compound of formula (I) in crystalline form characterized by substantially the same X-ray powder diffraction pattern shown in FIG. 10(a). In another embodiment, the compound is a mono-L-lactate salt of the compound of formula (I) in crystalline form characterized by substantially the same infrared spectrum shown in FIG. 10(b). In another embodiment, the compound is a mono-L-lactate salt of the compound of formula (I) in crystalline form characterized by substantially the same Raman spectrum shown in FIG. 10(b). In another embodiment, the mono-L-lactate salt of the compound of formula (I) in crystalline form is characterized by an X-ray powder diffraction pattern which substantially includes the peaks of Table X.

TABLE X

| | d- |
|---|---|
| Pos. [°2Th.] | spacing [Å] |
| 5.5 | 16.0 |
| 7.2 | 12.2 |
| 9.5 | 9.3 |
| 10.6 | 8.4 |
| 11.0 | 8.0 |
| 12.6 | 7.0 |
| 15.4 | 5.8 |
| 16.5 | 5.4 |
| 16.8 | 5.3 |
| 17.2 | 5.1 |
| 17.6 | 5.0 |
| 18.1 | 4.9 |
| 18.5 | 4.8 |
| 19.1 | 4.6 |
| 19.3 | 4.6 |
| 19.9 | 4.5 |
| 21.9 | 4.1 |
| 22.1 | 4.0 |
| 22.6 | 3.9 |
| 23.4 | 3.8 |
| 24.1 | 3.7 |
| 24.5 | 3.6 |
| 25.3 | 3.5 |
| 25.9 | 3.4 |
| 26.7 | 3.3 |
| 27.7 | 3.2 |

* Based on Cu Kα radiation.

In another embodiment, the mono-L-lactate salt of the compound of formula (I) in crystalline form is characterized by an X-ray powder diffraction pattern which at least substantially includes the XRPD °2θ peaks 5.5, 7.2, 9.5, 11.0 and 12.6.

In one embodiment, the salt of the compound of formula (I) is a citrate salt in non-solvated form, solvated form, or a mixture of non-solvated or solvated forms thereof.

In another embodiment, the salt of the compound of formula (I) is a monocitrate salt in non-solvated form, solvated form, or a mixture of non-solvated or solvated forms thereof. In one embodiment, the compound is a non-solvated form of the monocitrate salt of the compound of formula (I). In another embodiment, the compound is a solvated form of the monocitrate salt of the compound of formula (I). In one embodiment, the compound is a hydrated form of the monocitrate salt of the compound of formula (I). In another embodiment, the compound is an anhydrate form of the monocitrate salt of the compound of formula (I). In another embodiment, the compound is a mixture of non-solvated and solvated forms of the monocitrate salt of the compound of formula (I). In another embodiment, the compound is a mixture of hydrate and anhydrate forms of the monocitrate salt of the compound of formula (I).

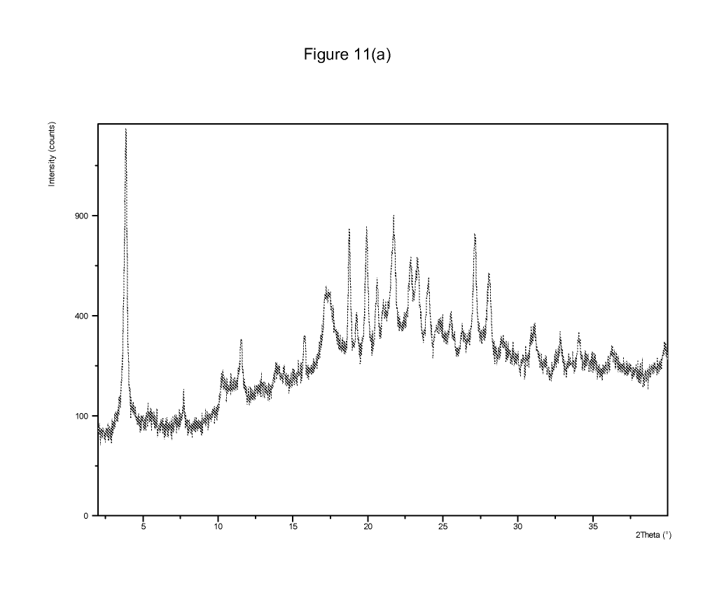

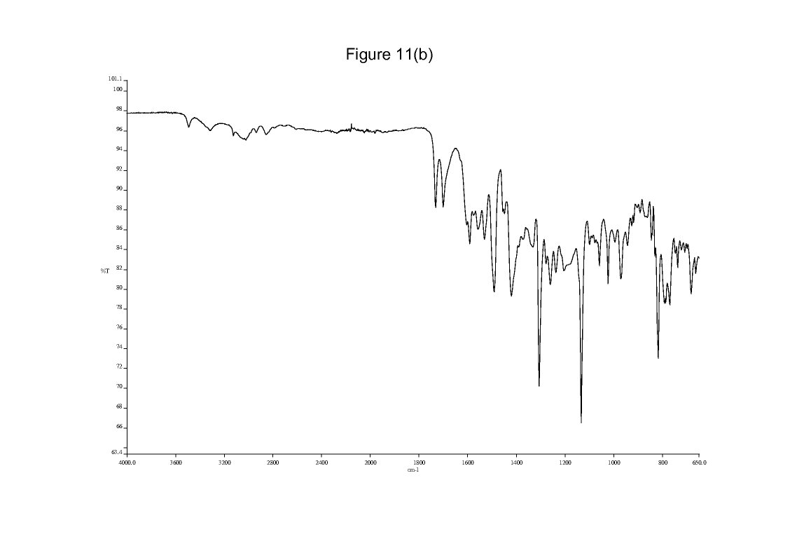

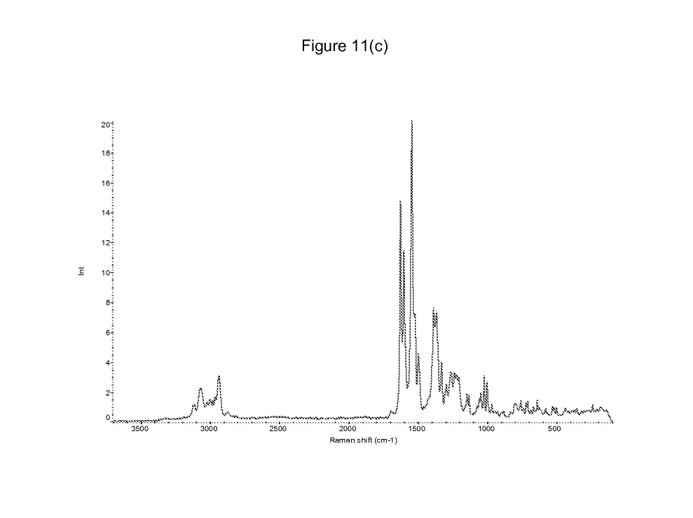

In another embodiment, the compound is a monocitrate salt of the compound of formula (I) in crystalline form characterized by substantially the same X-ray powder diffraction pattern shown in FIG. 11(a). In another embodiment, the compound is a monocitrate salt of the compound of formula (I) in crystalline form characterized by substantially the same infrared spectrum shown in FIG. 11(b). In another embodiment, the compound is a monocitrate salt of the compound of formula (I) in crystalline form characterized by substantially the same Raman spectrum shown in FIG. 11(b). In another embodiment, the monocitrate salt of the compound of formula (I) in crystalline form is characterized by an X-ray powder diffraction pattern which substantially includes the peaks of Table XI.

TABLE XI

| | d- |
|---|---|
| Pos. [°2Th.] | spacing [Å] |
| 3.9 | 22.8 |
| 7.7 | 11.5 |
| 11.6 | 7.6 |
| 15.8 | 5.6 |
| 17.2 | 5.2 |
| 18.8 | 4.7 |
| 19.2 | 4.6 |
| 19.9 | 4.5 |
| 20.6 | 4.3 |
| 21.0 | 4.2 |
| 21.8 | 4.1 |
| 22.9 | 3.9 |
| 23.3 | 3.8 |
| 24.1 | 3.7 |
| 25.5 | 3.5 |
| 27.2 | 3.3 |
| 28.1 | 3.2 |
| 31.1 | 2.9 |

* Based on Cu Kα radiation.

In another embodiment, the monocitrate salt of the compound of formula (I) in crystalline form is characterized by an X-ray powder diffraction pattern which at least substantially includes the XRPD °2θ peaks 3.9, 18.8, 19.9, 21.8 and 27.2.

In one embodiment, the salt of the compound of formula (I) is a benzoate salt in non-solvated form, solvated form, or a mixture of non-solvated or solvated forms thereof.

In another embodiment, the salt of the compound of formula (I) is a dibenzoate salt in non-solvated form, solvated form, or a mixture of non-solvated or solvated forms thereof. In one embodiment, the compound is a non-solvated form of the dibenzoate salt of the compound of formula (I). In another embodiment, the compound is a solvated form of the dibenzoate salt of the compound of formula (I). In one embodiment, the compound is a hydrated form of the dibenzoate salt of the compound of formula (I). In another embodiment, the compound is an anhydrate form of the dibenzoate salt of the compound of formula (I). In another embodiment, the compound is a mixture of non-solvated and solvated forms of the dibenzoate salt of the compound of formula (I). In another embodiment, the compound is a mixture of hydrate and anhydrate forms of the dibenzoate salt of the compound of formula (I).

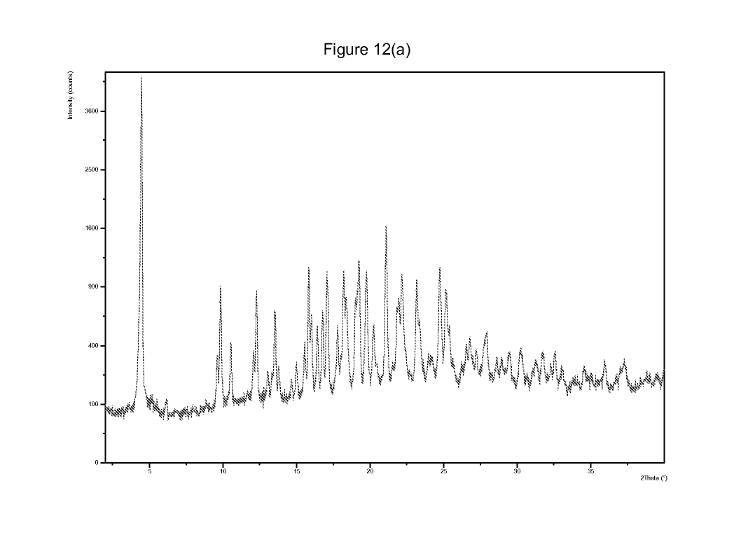

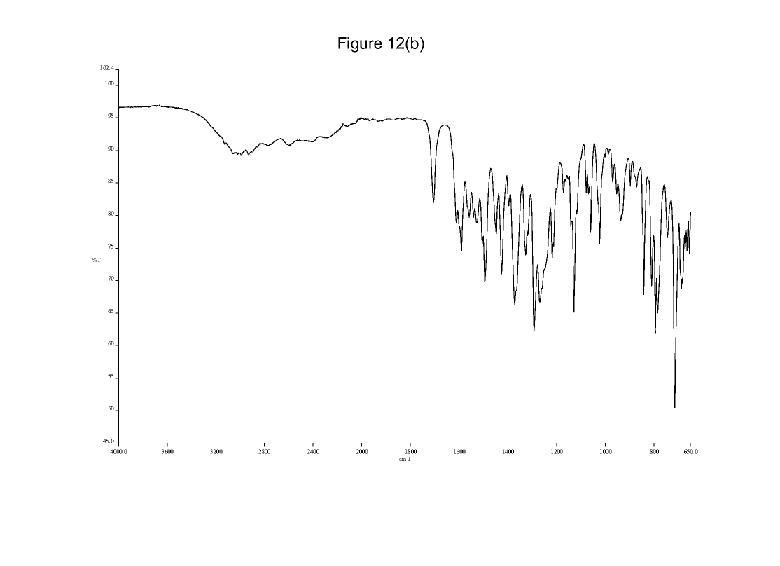

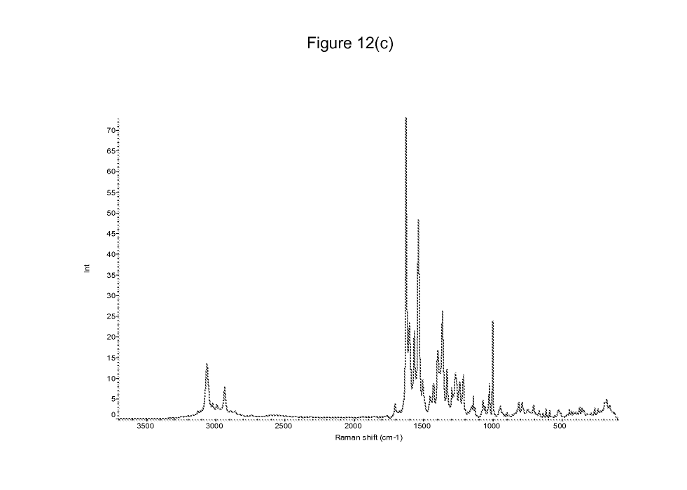

In another embodiment, the compound is a dibenzoate salt of the compound of formula (I) in crystalline form characterized by substantially the same X-ray powder diffraction pattern shown in FIG. 12(a). In another embodiment, the compound is a dibenzoate salt of the compound of formula (I) in crystalline form characterized by substantially the same infrared spectrum shown in FIG. 12(b). In another embodiment, the compound is a dibenzoate salt of the compound of formula (I) in crystalline form characterized by substantially the same Raman spectrum shown in FIG. 12(b). In another embodiment, the dibenzoate salt of the compound of formula (I) in crystalline form is characterized by an X-ray powder diffraction pattern which substantially includes the peaks of Table XII.

TABLE XII

| | d- |
|---|---|
| Pos. [°2Th.] | spacing [Å] |
| 4.5 | 19.9 |
| 9.6 | 9.2 |
| 9.9 | 9.0 |
| 10.5 | 8.4 |
| 12.1 | 7.3 |
| 12.3 | 7.2 |
| 13.0 | 6.8 |
| 13.5 | 6.5 |
| 13.8 | 6.4 |
| 14.6 | 6.1 |
| 15.0 | 5.9 |
| 15.5 | 5.7 |
| 15.8 | 5.6 |
| 16.0 | 5.5 |
| 16.4 | 5.4 |
| 16.8 | 5.3 |
| 17.1 | 5.2 |
| 17.8 | 5.0 |
| 18.2 | 4.9 |
| 18.4 | 4.8 |
| 19.0 | 4.7 |
| 19.2 | 4.6 |
| 19.7 | 4.5 |
| 20.2 | 4.4 |
| 21.1 | 4.2 |
| 21.8 | 4.1 |
| 22.0 | 4.0 |
| 22.2 | 4.0 |
| 23.2 | 3.8 |
| 24.7 | 3.6 |
| 25.1 | 3.5 |

* Based on Cu Kα radiation.

In another embodiment, the dibenzoate salt of the compound of formula (I) in crystalline form is characterized by an X-ray powder diffraction pattern which at least substantially includes the XRPD °2θ peaks 4.5, 9.9, 12.3, 19.7 and 21.1.

In one embodiment, the salt of the compound of formula (I) is a malate salt in non-solvated form, solvated form, or a mixture of non-solvated or solvated forms thereof.

In one embodiment, the salt of the compound of formula (I) is a monomalate salt in non-solvated form, solvated form, or a mixture of non-solvated or solvated forms thereof. In one embodiment, the compound is a non-solvated form of the monomalate salt of the compound of formula (I). In another embodiment, the compound is a solvated form of the monomalate salt of the compound of formula (I).

In one embodiment, the compound is a hydrated form of the monomalate salt of the compound of formula (I). In another embodiment, the compound is an anhydrate form of the monomalate salt of the compound of formula (I). In another embodiment, the compound is a mixture of non-solvated and solvated forms of the monomalate salt of the compound of formula (I). In another embodiment, the compound is a mixture of hydrate and anhydrate forms of the monomalate salt of the compound of formula (I).

In one embodiment, the salt of the compound of formula (I) is a mono-L-malate salt in non-solvated form, solvated form, or a mixture of non-solvated or solvated forms thereof. In one embodiment, the compound is a non-solvated form of the mono-L-malate salt of the compound of formula (I). In another embodiment, the compound is a solvated form of the mono-L-malate salt of the compound of formula (I). In one embodiment, the compound is a hydrated form of the mono-L-malate salt of the compound of formula (I). In another embodiment, the compound is an anhydrate form of the mono-L-malate salt of the compound of formula (I). In another embodiment, the compound is a mixture of non-solvated and solvated forms of the mono-L-malate salt of the compound of formula (I). In another embodiment, the compound is a mixture of hydrate and anhydrate forms of the mono-L-malate salt of the compound of formula (I).

In one embodiment, the salt of the compound of formula (I) is a dimalate salt in non-solvated form, solvated form, or a mixture of non-solvated or solvated forms thereof. In one embodiment, the compound is a non-solvated form of the dimalate salt of the compound of formula (I). In another embodiment, the compound is a solvated form of the dimalate salt of the compound of formula (I). In one embodiment, the compound is a hydrated form of the dimalate salt of the compound of formula (I). In another embodiment, the compound is an anhydrate form of the dimalate salt of the compound of formula (I). In another embodiment, the compound is a mixture of non-solvated and solvated forms of the dimalate salt of the compound of formula (I). In another embodiment, the compound is a mixture of hydrate and anhydrate forms of the dimalate salt of the compound of formula (I).

In one embodiment, the salt of the compound of formula (I) is a di-L-malate salt in non-solvated form, solvated form, or a mixture of non-solvated or solvated forms thereof. In one embodiment, the compound is a non-solvated form of the di-L-malate salt of the compound of formula (I). In another embodiment, the compound is a solvated form of the di-L-malate salt of the compound of formula (I). In one embodiment, the compound is a hydrated form of the di-L-malate salt of the compound of formula (I). In another embodiment, the compound is an anhydrate form of the di-L-malate salt of the compound of formula (I). In another embodiment, the compound is a mixture of non-solvated and solvated forms of the di-L-malate salt of the compound of formula (I). In another embodiment, the compound is a mixture of hydrate and anhydrate forms of the di-L-malate salt of the compound of formula (I).

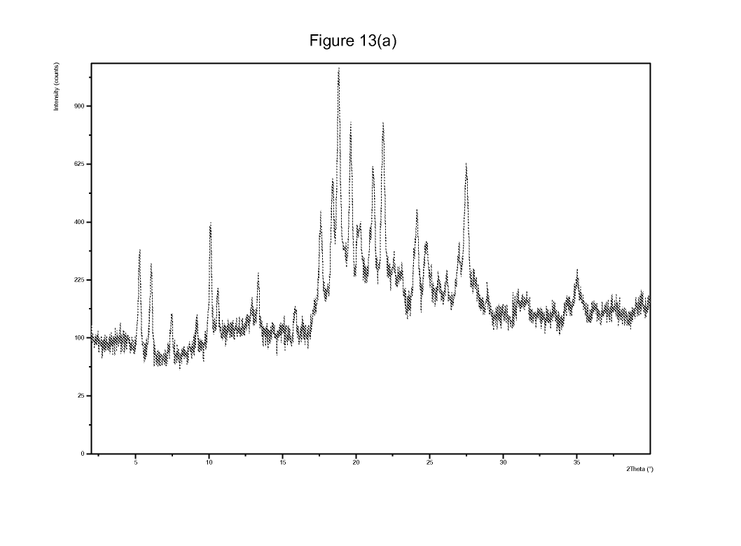

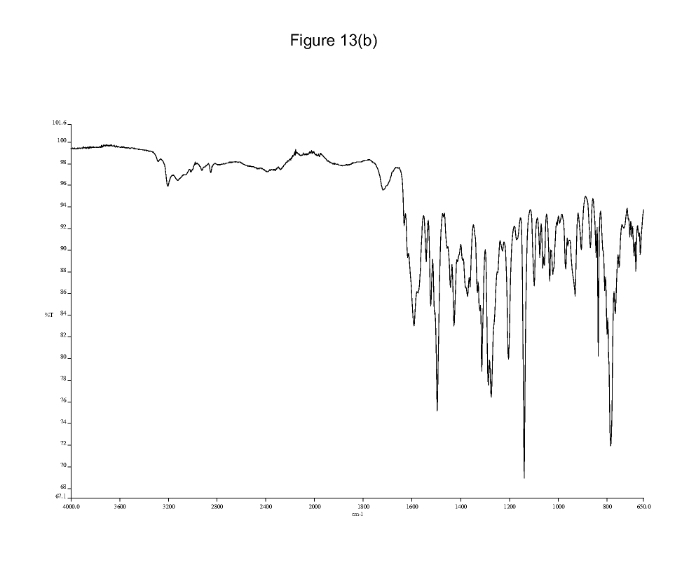

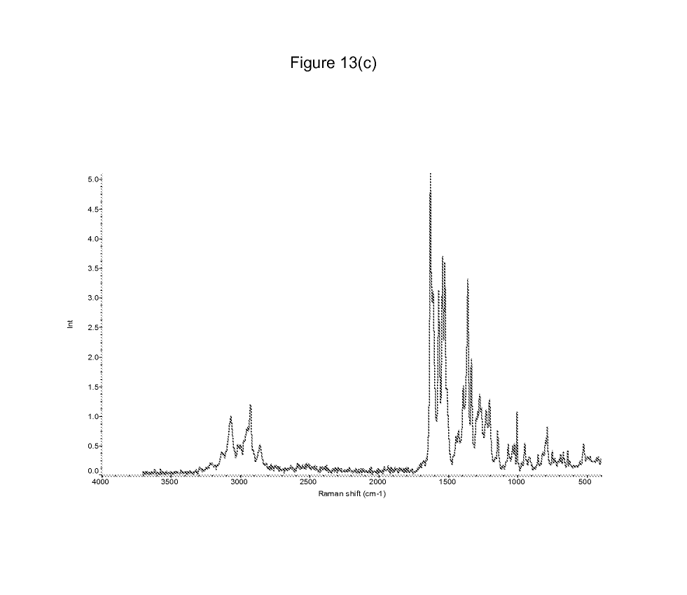

In another embodiment, the compound is a mono-L-malate salt of the compound of formula (I) in crystalline form characterized by substantially the same X-ray powder diffraction pattern shown in FIG. 13(a). In another embodiment, the compound is a mono-L-malate salt of the compound of formula (I) in crystalline form characterized by substantially the same infrared spectrum shown in FIG. 13(b). In another embodiment, the compound is a mono-L-malate salt of the compound of formula (I) in crystalline form characterized by substantially the same Raman spectrum shown in FIG. 13(b). In another embodiment, the mono-L-malate salt of the compound of formula (I) in crystalline form is characterized by an X-ray powder diffraction pattern which substantially includes the peaks of Table XIII.

TABLE XIII

| | d- |
|---|---|
| Pos. [°2Th.] | spacing [Å] |
| 5.3 | 16.8 |
| 6.1 | 14.6 |
| 7.5 | 11.8 |
| 9.2 | 9.6 |
| 10.1 | 8.8 |
| 10.6 | 8.3 |
| 12.9 | 6.9 |
| 13.4 | 6.6 |
| 15.9 | 5.6 |
| 17.6 | 5.0 |
| 18.5 | 4.8 |
| 18.8 | 4.7 |
| 19.6 | 4.5 |
| 20.1 | 4.4 |
| 20.3 | 4.4 |
| 21.1 | 4.2 |
| 21.9 | 4.1 |
| 23.1 | 3.8 |
| 24.2 | 3.7 |
| 24.8 | 3.6 |
| 25.6 | 3.5 |
| 26.2 | 3.4 |
| 27.0 | 3.3 |
| 27.5 | 3.2 |

*Based on Cu Kα radiation

In another embodiment, the mono-L-malate salt of the compound of formula (I) in crystalline form is characterized by an X-ray powder diffraction pattern which at least substantially includes the XRPD °2θ peaks 5.3, 6.1, 10.1, 19.6 and 21.90.

In a further embodiment, the salt of the compound of formula (I) is a hydrobromide or tartrate salt in non-solvated form, solvated form, or a mixture of non-solvated or solvated forms thereof.

In a further embodiment, the salt of the compound of formula (I) is a di-L-tartrate, mono-L-tartrate, or dihydrobromide salt in non-solvated form, solvated form, or a mixture of non-solvated or solvated forms thereof.

In one embodiment, the salt of the compound of formula (I) is a hydrobromide salt in non-solvated form, solvated form, or a mixture of non-solvated or solvated forms thereof. In one embodiment, the compound is a non-solvated form of the hydrobromide salt of the compound of formula (I). In another embodiment, the compound is a solvated form of the hydrobromide salt of the compound of formula (I). In one embodiment, the compound is a hydrated form of the hydrobromide salt of the compound of formula (I). In another embodiment, the compound is an anhydrate form of the hydrobromide salt of the compound of formula (I). In another embodiment, the compound is a mixture of non-solvated and solvated forms of the hydrobromide salt of the compound of formula (I). In another embodiment, the compound is a mixture of hydrate and anhydrate forms of the hydrobromide salt of the compound of formula (I).

In one embodiment, the salt of the compound of formula (I) is a tartrate salt in non-solvated form, solvated form, or a mixture of non-solvated or solvated forms thereof.

In one embodiment, the salt of the compound of formula (I) is a monotartrate salt in non-solvated form, solvated form, or a mixture of non-solvated or solvated forms thereof. In one embodiment, the compound is a non-solvated form of the monotartrate salt of the compound of formula (I). In another embodiment, the compound is a solvated form of the monotartrate salt of the compound of formula (I). In one embodiment, the compound is a hydrated form of the monotartrate salt of the compound of formula (I). In another embodiment, the compound is an anhydrate form of the monotartrate salt of the compound of formula (I). In another embodiment, the compound is a mixture of non-solvated and solvated forms of the monotartrate salt of the compound of formula (I). In another embodiment, the compound is a mixture of hydrate and anhydrate forms of the monotartrate salt of the compound of formula (I).

In one embodiment, the salt of the compound of formula (I) is a mono-L-tartrate salt in non-solvated form, solvated form, or a mixture of non-solvated or solvated forms thereof. In one embodiment, the compound is a non-solvated form of the mono-L-tartrate salt of the compound of formula (I). In another embodiment, the compound is a solvated form of the mono-L-tartrate salt of the compound of formula (I). In one embodiment, the compound is a hydrated form of the mono-L-tartrate salt of the compound of formula (I). In another embodiment, the compound is an anhydrate form of the mono-L-tartrate salt of the compound of formula (I). In another embodiment, the compound is a mixture of non-solvated and solvated forms of the mono-L-tartrate salt of the compound of formula (I). In another embodiment, the compound is a mixture of hydrate and anhydrate forms of the mono-L-tartrate salt of the compound of formula (I).

In one embodiment, the salt of the compound of formula (I) is a ditartrate salt in non-solvated form, solvated form, or a mixture of non-solvated or solvated forms thereof. In one embodiment, the compound is a non-solvated form of the ditartrate salt of the compound of formula (I). In another embodiment, the compound is a solvated form of the ditartrate salt of the compound of formula (I). In one embodiment, the compound is a hydrated form of the ditartrate salt of the compound of formula (I). In another embodiment, the compound is an anhydrate form of the ditartrate salt of the compound of formula (I). In another embodiment, the compound is a mixture of non-solvated and solvated forms of the ditartrate salt of the compound of formula (I). In another embodiment, the compound is a mixture of hydrate and anhydrate forms of the ditartrate salt of the compound of formula (I).

In one embodiment, the salt of the compound of formula (I) is a di-L-tartrate salt in non-solvated form, solvated form, or a mixture of non-solvated or solvated forms thereof. In one embodiment, the compound is a non-solvated form of the di-L-tartrate salt of the compound of formula (I). In another embodiment, the compound is a solvated form of the di-L-tartrate salt of the compound of formula (I). In one embodiment, the compound is a hydrated form of the di-L-tartrate salt of the compound of formula (I). In another embodiment, the compound is an anhydrate form of the di-L-tartrate salt of the compound of formula (I). In another embodiment, the compound is a mixture of non-solvated and solvated forms of the di-L-tartrate salt of the compound of formula (I). In another embodiment, the compound is a mixture of hydrate and anhydrate forms of the di-L-tartrate salt of the compound of formula (I).

In another embodiment, the salt of the compound of formula (I) is a fumarate, benzensulfonate (besylate), salicylate, succinate, or ethanedisulfonate (edisylate) in non-solvated form, solvated form, or a mixture of non-solvated or solvated forms thereof.

In one embodiment, the salt of the compound of formula (I) is a succinate salt in non-solvated form, solvated form, or a mixture of non-solvated or solvated forms thereof.

In another embodiment, the salt of the compound of formula (I) is a monosuccinate salt in non-solvated form, solvated form, or a mixture of non-solvated or solvated forms thereof. In one embodiment, the compound is a non-solvated form of the monosuccinate salt of the compound of formula (I). In another embodiment, the compound is a solvated form of the monosuccinate salt of the compound of formula (I). In one embodiment, the compound is a hydrated form of the monosuccinate salt of the compound of formula (I). In another embodiment, the compound is an anhydrate form of the monosuccinate salt of the compound of formula (I). In another embodiment, the compound is a mixture of non-solvated and solvated forms of the monosuccinate salt of the compound of formula (I). In another embodiment, the compound is a mixture of hydrate and anhydrate forms of the monosuccinate salt of the compound of formula (I).

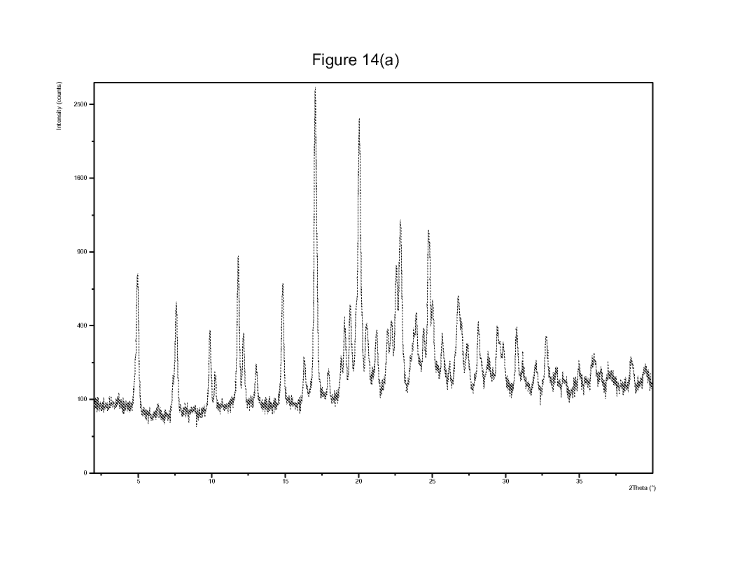

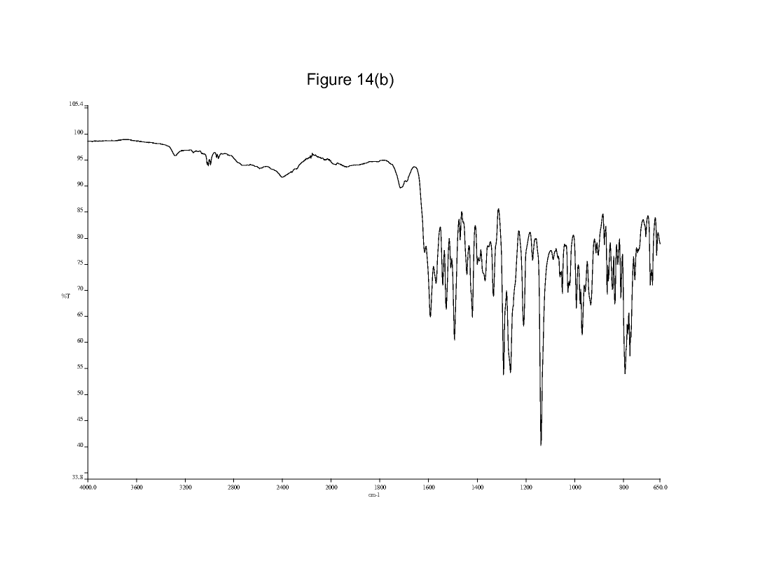

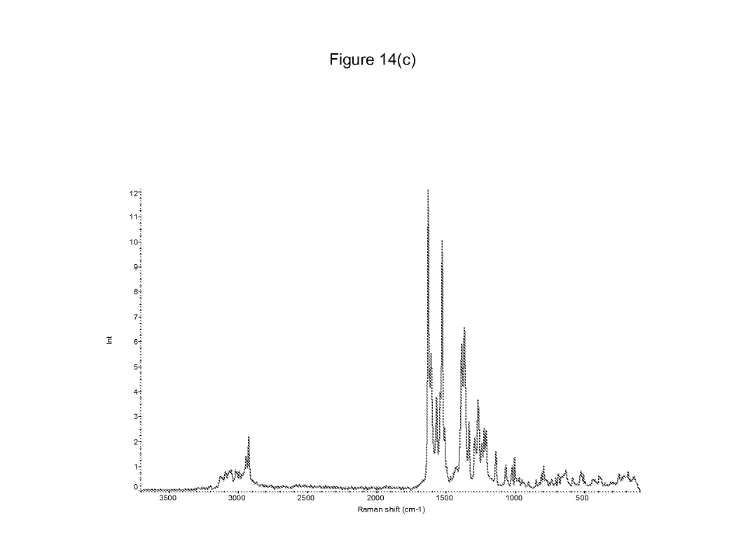

In another embodiment, the compound is a monosuccinate salt of the compound of formula (I) in crystalline form characterized by substantially the same X-ray powder diffraction pattern shown in FIG. 14(a). In another embodiment, the compound is a monosuccinate salt of the compound of formula (I) in crystalline form characterized by substantially the same infrared spectrum shown in FIG. 14(b). In another embodiment, the compound is a monosuccinate salt of the compound of formula (I) in crystalline form characterized by substantially the same Raman spectrum shown in FIG. 14(b). In another embodiment, the monosuccinate salt of the compound of formula (I) in crystalline form is characterized by an X-ray powder diffraction pattern which substantially includes the peaks of Table XIV.

TABLE XIV

| | d- |
|---|---|
| Pos. [°2Th.] | spacing [Å] |
| 5.0 | 17.8 |
| 7.6 | 11.7 |
| 9.9 | 9.0 |
| 10.2 | 8.6 |
| 11.8 | 7.5 |
| 12.2 | 7.3 |
| 13.0 | 6.8 |
| 14.8 | 6.0 |
| 16.3 | 5.4 |
| 17.0 | 5.2 |
| 17.9 | 4.9 |
| 18.8 | 4.7 |
| 19.0 | 4.7 |
| 19.4 | 4.6 |
| 20.0 | 4.4 |
| 20.5 | 4.3 |
| 21.2 | 4.2 |
| 22.0 | 4.0 |
| 22.2 | 4.0 |
| 22.5 | 3.9 |
| 22.8 | 3.9 |
| 23.9 | 3.7 |
| 24.4 | 3.7 |
| 24.8 | 3.6 |
| 25.1 | 3.6 |
| 26.8 | 3.3 |
| 27.0 | 3.3 |
| 28.1 | 3.2 |
| 29.4 | 3.0 |

*Based on Cu Kα radiation

In another embodiment, the monosuccinate salt of the compound of formula (I) in crystalline form is characterized by an X-ray powder diffraction pattern which at least substantially includes the XRPD °2θ peaks 5.0, 7.6, 11.8, 14.8 and 17.0.

In one embodiment, the salt of the compound of formula (I) is a benzenesulfonate (besylate) salt in non-solvated form, solvated form, or a mixture of non-solvated or solvated forms thereof.

In one embodiment, the salt of the compound of formula (I) is a dibesylate salt in non-solvated form, solvated form, or a mixture of non-solvated or solvated forms thereof. In one embodiment, the compound is a non-solvated form of the dibesylate salt of the compound of formula (I). In another embodiment, the compound is a solvated form of the dibesylate salt of the compound of formula (I). In one embodiment, the compound is a hydrated form of the dibesylate salt of the compound of formula (I). In another embodiment, the compound is an anhydrate form of the dibesylate salt of the compound of formula (I). In another embodiment, the compound is a mixture of non-solvated and solvated forms of the dibesylate salt of the compound of formula (I). In another embodiment, the compound is a mixture of hydrate and anhydrate forms of the dibesylate salt of the compound of formula (I).

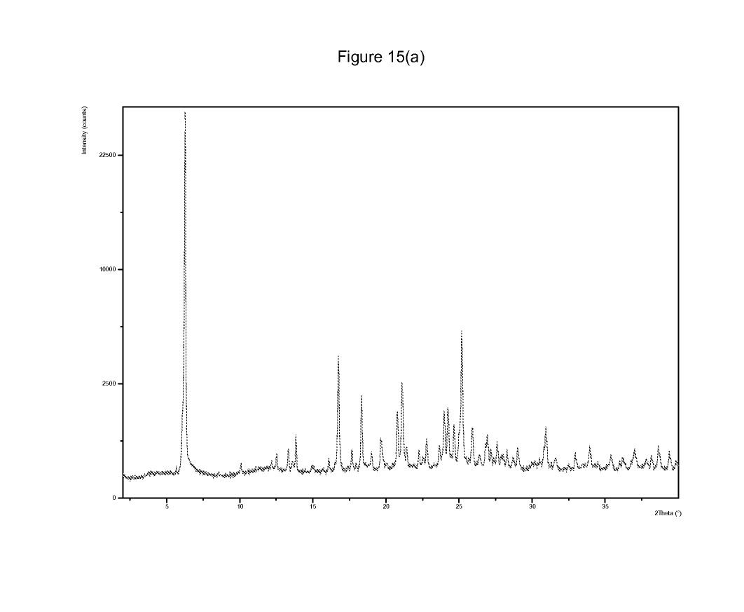

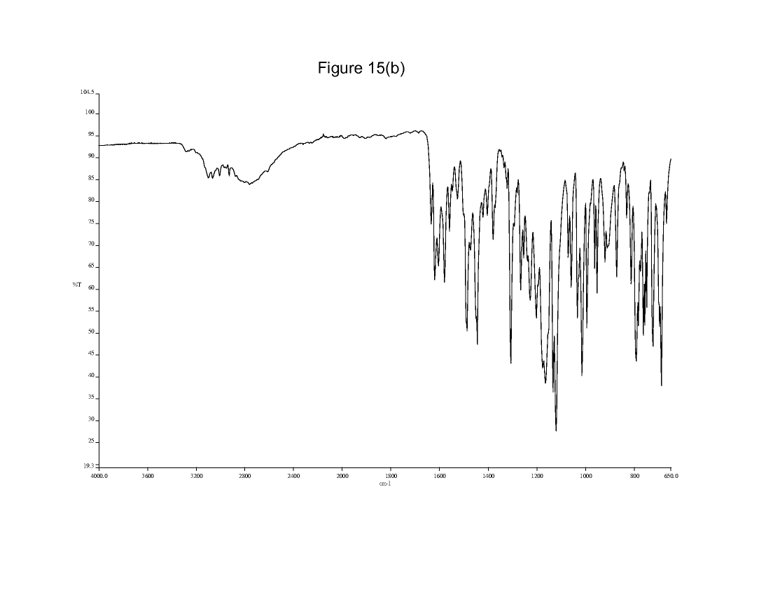

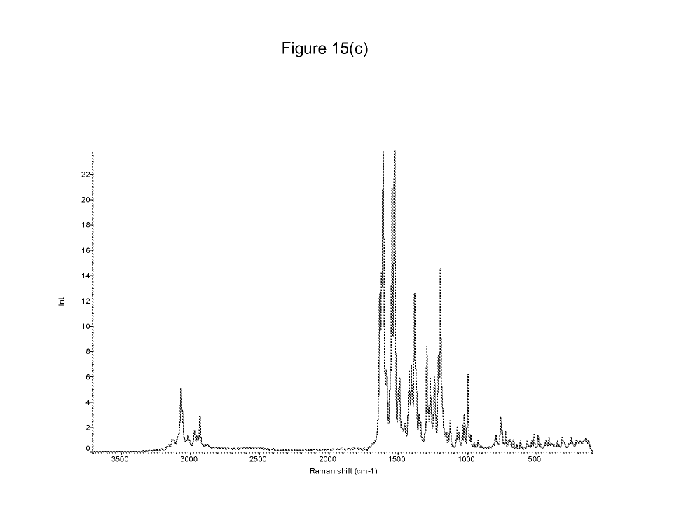

In another embodiment, the compound is a dibesylate salt of the compound of formula (I) in crystalline form characterized by substantially the same X-ray powder diffraction pattern shown in FIG. 15(a). In another embodiment, the compound is a dibesylate salt of the compound of formula (I) in crystalline form characterized by substantially the same infrared spectrum shown in FIG. 15(b). In another embodiment, the compound is a dibesylate salt of the compound of formula (I) in crystalline form characterized by substantially the same Raman spectrum shown in FIG. 15(b). In another embodiment, the dibesylate salt of the compound of formula (I) in crystalline form is characterized by an X-ray powder diffraction pattern which substantially includes the peaks of Table XV.

TABLE XV

| | d- |
|---|---|
| Pos. [°2Th.] | spacing [Å] |
| 6.3 | 14.1 |
| 10.1 | 8.8 |
| 12.2 | 7.3 |
| 12.5 | 7.1 |
| 13.3 | 6.6 |
| 13.6 | 6.5 |
| 13.8 | 6.4 |
| 16.1 | 5.5 |
| 16.8 | 5.3 |
| 17.7 | 5.0 |
| 18.3 | 4.8 |
| 19.0 | 4.7 |
| 19.6 | 4.5 |
| 20.8 | 4.3 |
| 21.1 | 4.2 |
| 21.4 | 4.2 |
| 22.8 | 3.9 |
| 23.7 | 3.8 |
| 24.0 | 3.7 |
| 24.2 | 3.7 |
| 24.6 | 3.6 |
| 25.0 | 3.6 |
| 25.2 | 3.5 |
| 25.9 | 3.4 |
| 26.8 | 3.3 |
| 26.9 | 3.3 |
| 27.6 | 3.2 |
| 30.9 | 2.9 |
| 33.9 | 2.6 |

*Based on Cu Kα radiation

In another embodiment, the dibesylate salt of the compound of formula (I) in crystalline form is characterized by an X-ray powder diffraction pattern which at least substantially includes the XRPD °2θ peaks 6.3, 16.8, 18.3, 21.1 and 25.2.

In one embodiment, the salt of the compound of formula (I) is a fumarate salt in non-solvated form, solvated form, or a mixture of non-solvated or solvated forms thereof.

In one embodiment, the salt of the compound of formula (I) is a monofumarate salt in non-solvated form, solvated form, or a mixture of non-solvated or solvated forms thereof. In one embodiment, the compound is a non-solvated form of the monofumarate salt of the compound of formula (I). In another embodiment, the compound is a solvated form of the monofumarate salt of the compound of formula (I). In one embodiment, the compound is a hydrated form of the monofumarate salt of the compound of formula (I). In another embodiment, the compound is an anhydrate form of the monofumarate salt of the compound of formula (I). In another embodiment, the compound is a mixture of non-solvated and solvated forms of the monofumarate salt of the compound of formula (I). In another embodiment, the compound is a mixture of hydrate and anhydrate forms of the monofumarate salt of the compound of formula (I).

In one embodiment, the salt of the compound of formula (I) is a difumarate salt in non-solvated form, solvated form, or a mixture of non-solvated or solvated forms thereof. In one embodiment, the compound is a non-solvated form of the difumarate salt of the compound of formula (I). In another embodiment, the compound is a solvated form of the difumarate salt of the compound of formula (I). In one embodiment, the compound is a hydrated form of the difumarate salt of the compound of formula (I). In another embodiment, the compound is an anhydrate form of the difumarate salt of the compound of formula (I). In another embodiment, the compound is a mixture of non-solvated and solvated forms of the difumarate salt of the compound of formula (I). In another embodiment, the compound is a mixture of hydrate and anhydrate forms of the difumarate salt of the compound of formula (I).

In one embodiment, the salt of the compound of formula (I) is a 1,2-ethanedisulfonate (edisylate) salt or non-solvated or solvated forms thereof.

In one embodiment, the salt of the compound of formula (I) is an hemi-edisylate salt in non-solvated form, solvated form, or a mixture of non-solvated or solvated forms thereof. In one embodiment, the compound is a non-solvated form of the hemi-edisylate salt of the compound of formula (I). In another embodiment, the compound is a solvated form of the hemi-edisylate salt of the compound of formula (I). In one embodiment, the compound is a hydrated form of the hemi-edisylate salt of the compound of formula (I). In another embodiment, the compound is an anhydrate form of the hemi-edisylate salt of the compound of formula (I). In another embodiment, the compound is a mixture of non-solvated and solvated forms of the hemi-edisylate salt of the compound of formula (I). In another embodiment, the compound is a mixture of hydrate and anhydrate forms of the hemi-edisylate salt of the compound of formula (I).

In one embodiment, the salt of the compound of formula (I) is a salicylate salt in non-solvated form, solvated form, or a mixture of non-solvated or solvated forms thereof.

In one embodiment, the salt of the compound of formula (I) is a monosalicylate salt in non-solvated form, solvated form, or a mixture of non-solvated or solvated forms thereof. In one embodiment, the compound is a non-solvated form of the monosalicylate salt of the compound of formula (I). In another embodiment, the compound is a solvated form of the monosalicylate salt of the compound of formula (I). In one embodiment, the compound is a hydrated form of the monosalicylate salt of the compound of formula (I). In another embodiment, the compound is an anhydrate form of the monosalicylate salt of the compound of formula (I). In another embodiment, the compound is a mixture of non-solvated and solvated forms of the monosalicylate salt of the compound of formula (I). In another embodiment, the compound is a mixture of hydrate and anhydrate forms of the monosalicylate salt of the compound of formula (I).

In one embodiment, each of the salts of the compound of formula (I) described above are pharmaceutically acceptable salts.

While it is possible that, for use in therapy, therapeutically effective amounts of a salt of the compound of formula (I), as well as a non-solvated form, solvated form, or a mixture of non-solvated or solvated forms thereof, may be administered as the raw chemical, it is possible to present the active ingredient as a pharmaceutical composition. Accordingly, the invention further provides pharmaceutical compositions, which include therapeutically effective amounts of a salt of the compounds of the formula (I) in non-solvated form, solvated form, or a mixture of non-solvated or solvated forms thereof, and one or more pharmaceutically acceptable carriers, diluents, or excipients. The salts of the compounds of the formula (I) in non-solvated form, solvated form, or a mixture of non-solvated or solvated forms thereof, are as described above. The carrier(s), diluent(s) or excipient(s) must be acceptable in the sense of being compatible with the other ingredients of the formulation and not deleterious to the recipient thereof. According to another aspect of the invention there is also provided a process for the preparation of a pharmaceutical formulation including admixing a compound of the formula (I), in non-solvated form, solvated form, or a mixture of non-solvated or solvated forms thereof, with one or more pharmaceutically acceptable carriers, diluents or excipients.

Salts of the compounds of formula (I) in non-solvated form, solvated form, or a mixture of non-solvated or solvated forms thereof may be formulated for administration by any route, and the appropriate route will depend on the disease being treated as well as the subjects to be treated. Suitable pharmaceutical formulations include those for oral, rectal, nasal, topical (including buccal, sub-lingual, and transdermal), vaginal or parenteral (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well know in the pharmacy art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agents can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The salts of the compound of formula (I), in non-solvated form, solvated form, or a mixture of non-solvated or solvated forms thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The salts of the compound of formula (I) in non-solvated form, solvated form, or a mixture of non-solvated or solvated forms thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation, through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In one embodiment, there is provided a pharmaceutical composition including a therapeutically effective amount of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof. In another embodiment, the pharmaceutical composition further includes one or more pharmaceutically acceptable, carriers, diluents and excipients.

In one embodiment, there is provided a pharmaceutical composition including a therapeutically effective amount of a salt of a compound of formula (I) wherein said salt is an ethanesulfonate (esylate), methanesulfonate (mesylate), lactate, malate, maleate, benzoate, or citrate salt in non-solvated form, solvated form, or a mixture of non-solvated or solvated forms thereof. In another embodiment, the pharmaceutical composition further includes one or more pharmaceutically acceptable, carriers, diluents and excipients.

In one embodiment, there is provided a pharmaceutical composition including a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof. In another embodiment, the pharmaceutical composition further includes one or more pharmaceutically acceptable, carriers, diluents and excipients.

In one embodiment, there is provided a pharmaceutical composition including a therapeutically effective amount of a salt of a compound of formula (I) wherein said salt is a hydrobromide or tartrate salt in non-solvated form, solvated form, or a mixture of non-solvated or solvated forms thereof. In another embodiment, the pharmaceutical composition further includes one or more pharmaceutically acceptable, carriers, diluents and excipients.

In one embodiment, there is provided a pharmaceutical composition including a therapeutically effective amount of a salt of a compound of formula (I) wherein said salt is a fumarate, benzensulfonate, salicylate, succinate, or ethanedisulfonate salt in non-solvated form, solvated form, or a mixture of non-solvated or solvated forms thereof. In another embodiment, the pharmaceutical composition further includes one or more pharmaceutically acceptable, carriers, diluents and excipients.

The salts of the compound of formula (I) and pharmaceutically acceptable carriers, diluents, and excipients are as described above.

Also provided in the present invention, is a method for treating a disorder in a mammal characterized by aberrant activity of at least one erbB family protein tyrosine kinase (PTK) which includes administering a therapeutically effective amount of a salt of the compound of formula (I) in non-solvated form, solvated form, or a mixture of non-solvated or solvated forms thereof, to the mammal. The salts of the compound of formula (I) in non-solvated form, solvated form, or a mixture of non-solvated or solvated forms thereof are as described above.

The aberrant PTK activity referred to herein is any erbB family PTK activity that deviates from the normal erbB family protein kinase activity expected in a particular mammalian subject. Aberrant erbB family PTK activity may take the form of, for instance, an abnormal increase in activity, or an aberration in the timing and or control of PTK activity. Such aberrant activity may result then, for example, from overexpression or mutation of the protein kinase leading to inappropriate or uncontrolled activation. Furthermore, it is also understood that unwanted PTK activity may reside in an abnormal source, such as a malignancy. That is, the level of PTK activity does not have to be abnormal to be considered aberrant, rather the activity derives from an abnormal source.

The salt of the compound of formula (I) in non-solvated form, solvated form, or a mixture of non-solvated or solvated forms thereof, are inhibitors of one or more erbB family PTKs and as such have utility in the treatment of disorders in mammals which are characterized by aberrant PTK activity, particularly humans. In one embodiment of the present invention, the disorder treated is characterized by at least one erbB family PTK, selected from EGFr, erbB-2 and erbB-4, exhibiting aberrant activity. In another embodiment, the disorder treated is characterized by at least two erbB family PTKs, selected from EGFr, erbB-2 and erbB-4, exhibiting aberrant activity. In one embodiment of the treatment method, the salt of the compound of formula (I) in non-solvated form, solvated form, or a mixture of non-solvated or solvated forms thereof inhibit at least one erbB family PTK, selected from EGFr, erbB-2 and erbB-4. In another embodiment of the treatment method, the salt of the compound of formula I or non-solvated form, or solvated form, or a mixture of non-solvated or solvated forms thereof inhibit at least two erbB family PTKs selected from EGFr, c-erb-B2 and c-erb-B4. In one embodiment, there is provided a method of inhibiting at least one of EGFr, erbB-2 and erbB-4 in a mammal, the method including administering a therapeutically effective amount of a salt of the compound of formula (I) in non-solvated form, solvated form, or a mixture of non-solvated or solvated forms thereof. In another embodiment, there is provided a method of inhibiting at least two of EGFr, erbB-2 and erbB-4 in a mammal, the method including administering a therapeutically effective amount of a salt of the compound of formula (I) in non-solvated form, solvated form, or a mixture of non-solvated or solvated forms thereof.

Accordingly, also provided is a method of treating a disorder mediated by aberrant protein tyrosine kinase activity in a mammal, including: administering to said mammal an amount of a salt of the compound of formula (I) in non-solvated form, solvated form, or a mixture of non-solvated or solvated forms thereof, effective to inhibit at least one erbB family protein. In one embodiment, the method includes administering an amount of a salt of the compound of formula (I) in non-solvated form, solvated form, or a mixture of non-solvated or solvated forms thereof, effective to inhibit at least two erbB family proteins.

The disorders referred to may be any disorder which is characterized by aberrant PTK activity. As recited above such disorders include, but are not limited to, cancer and psoriasis. In one embodiment, the disorder is cancer. In another embodiment, the cancer is non-small cell lung, colo-rectal, bladder, prostate, liver, brain, head and neck, breast, renal, cervical, ovarian, gastric, esophageal, colorectal, or pancreatic cancers.

In one embodiment, there is provided a method of treating a cancer in a mammal, said cancer characterized by expression of at least one of EGFR, erbB-2 or erbB-4, comprising: administering to said mammal a therapeutically effective amount of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof.

In one embodiment, there is provided a method of treating a cancer in a mammal, said cancer characterized by expression of at least one of EGFR, erbB-2 or erbB-4, comprising: administering to said mammal a therapeutically effective amount of a salt of a compound of formula (I) wherein said salt is an ethanesulfonate methanesulfonate, lactate, malate, maleate, benzoate, or citrate salt in non-solvated form, solvated form, or a mixture of non-solvated or solvated forms, or a mixture of non-solvated or solvated forms thereof.

In one embodiment, there is provided a method of treating a cancer in a mammal, said cancer characterized by expression of at least one of EGFR, erbB-2 or erbB-4, comprising: administering to said mammal a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.

In one embodiment, there is provided a method of treating a cancer in a mammal, said cancer characterized by expression of at least one of EGFR, erbB-2 or erbB-4, comprising: administering to said mammal a therapeutically effective amount of a salt of a compound of formula (I) wherein said salt is a hydrobromide or tartrate salt in non-solvated form, solvated form, or a mixture of non-solvated or solvated forms thereof.

In one embodiment, there is provided a method of treating a cancer in a mammal, said cancer characterized by expression of at least one of EGFR, erbB-2 or erbB-4, comprising: administering to said mammal a therapeutically effective amount of a salt of a compound of formula (I) wherein said salt is a fumarate, benzensulfonate, salicylate, succinate, or ethanedisulfonate salt in non-solvated form, solvated form, or a mixture of non-solvated or solvated forms thereof.

In one embodiment, there is provided a method of treating a cancer in a mammal, said cancer characterized by expression of at least two of EGFR, erbB-2 or erbB-4, comprising: administering to said mammal a therapeutically effective amount of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof.

In one embodiment, there is provided a method of treating a cancer in a mammal, said cancer characterized by expression of at least two of EGFR, erbB-2 or erbB-4, comprising: administering to said mammal a therapeutically effective amount of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof.

In one embodiment, there is provided a method of treating a cancer in a mammal, said cancer characterized by expression of at least two of EGFR, erbB-2 or erbB-4, comprising: administering to said mammal a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.

In one embodiment, there is provided a method of treating a cancer in a mammal, said cancer characterized by expression of at least two of EGFR, erbB-2 or erbB-4, comprising: administering to said mammal a therapeutically effective amount of a salt of a compound of formula (I) wherein said salt is a hydrobromide or tartrate salt in non-solvated form, solvated form, or a mixture of non-solvated or solvated forms thereof.

In one embodiment, there is provided a method of treating a cancer in a mammal, said cancer characterized by expression of at least two of EGFR, erbB-2 or erbB-4, comprising: administering to said mammal a therapeutically effective amount of a salt of a compound of formula (I) wherein said salt is a fumarate, benzensulfonate, salicylate, succinate, or ethanedisulfonate salt in non-solvated form, solvated form, or a mixture of non-solvated or solvated forms thereof.

The salts of the compound of formula (I) are as described above.

In one embodiment, there is provided a method of inhibiting at least one of EGFR, erbB-2 or erbB-4 in a mammal, comprising: administering to said mammal a therapeutically effective amount of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof.

In one embodiment, there is provided a method of inhibiting at least one of EGFR, erbB-2 or erbB-4 in a mammal, comprising: administering to said mammal a therapeutically effective amount of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof.

In one embodiment, there is provided a method of inhibiting at least one of EGFR, erbB-2 or erbB-4 in a mammal, comprising: administering to said mammal a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.

In one embodiment, there is provided a method of inhibiting at least one of EGFR, erbB-2 or erbB-4 in a mammal, comprising: administering to said mammal a therapeutically effective amount of a salt of a compound of formula (I) wherein said salt is a hydrobromide or tartrate salt in non-solvated form, solvated form, or a mixture of non-solvated or solvated forms thereof.

In one embodiment, there is provided a method of inhibiting at least one of EGFR, erbB-2 or erbB-4, comprising: administering to said mammal a therapeutically effective amount of a salt of a compound of formula (I) wherein said salt is a fumarate, benzensulfonate, salicylate, succinate, or ethanedisulfonate salt in non-solvated form, solvated form, or a mixture of non-solvated or solvated forms thereof.

In one embodiment, there is provided a method of inhibiting at least two of EGFR, erbB-2 or erbB-4 in a mammal, comprising: administering to said mammal a therapeutically effective amount of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof.

In one embodiment, there is provided a method of inhibiting at least two of EGFR, erbB-2 or erbB-4 in a mammal, comprising: administering to said mammal a therapeutically effective amount of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof.

In one embodiment, there is provided a method of inhibiting at least two of EGFR, erbB-2 or erbB-4 in a mammal, comprising: administering to said mammal a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.

In one embodiment, there is provided a method of inhibiting at least two of EGFR, erbB-2 or erbB-4 in a mammal, comprising: administering to said mammal a therapeutically effective amount of a salt of a compound of formula (I) wherein said salt is a hydrobromide or tartrate salt in non-solvated form, solvated form, or a mixture of non-solvated or solvated forms thereof.

In one embodiment, there is provided a method of inhibiting at least two of EGFR, erbB-2 or erbB-4, comprising: administering to said mammal a therapeutically effective amount of a salt of a compound of formula (I) wherein said salt is a fumarate, benzensulfonate, salicylate, succinate, or ethanedisulfonate salt in non-solvated form, solvated form, or a mixture of non-solvated or solvated forms thereof.

In one embodiment, there is provided a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof for use in therapy.

In another embodiment, there is provided a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof for use in therapy.

In another embodiment, there is provided a salt of a compound of formula (I) wherein said salt is an diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof for use in therapy.

In another embodiment, there is provided a salt of a compound of formula (I) wherein said salt is a hydrobromide or tartrate salt in non-solvated form, solvated form, or a mixture of non-solvated or solvated forms thereof for use in therapy.

In another embodiment, there is provided a salt of a compound of formula (I) wherein said salt is an fumarate, benzensulfonate, salicylate, succinate, or ethanedisulfonate salt in non-solvated form, solvated form, or a mixture of non-solvated or solvated forms thereof for use in therapy.

The salts of the compound of formula (I) are as described above.

The salts of a compound of formula (I) in non-solvated form, solvated form, or a mixture of non-solvated or solvated forms thereof, described above, are useful in therapy and in the preparation of medicaments for treating a disorder in a mammal, which is characterized by aberrant activity of at least one erbB family PTK. In one embodiment of the present invention, the medicament prepared is useful in treating a disorder characterized by at least one erbB family PTK, selected from EGFr, c-erb-B2 and c-erb-B4, exhibiting aberrant activity. In another embodiment, the medicament prepared is useful in treating a disorder characterized by at least two erbB family PTKs, selected from EGFr, c-erb-B2 and c-erb-B4, exhibiting aberrant activity. In one embodiment of the use, the salt of the compound of formula (I) in non-solvated form, solvated form, or a mixture of non-solvated or solvated forms thereof, which are used to form the medicament, inhibit at least one erbB family PTK, selected from EGFr, c-erb-B2 and c-erb-B4. In another embodiment of the use, the salt of the compound of formula (I) in non-solvated form, solvated form, or a mixture of non-solvated or solvated forms thereof, which are used to form the medicament, inhibit at least two erbB family PTKs selected from EGFr, c-erb-B2 and c-erb-B4. The salts of the compound of formula (I) are as described above.

In one embodiment, there is provided use of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof in the preparation of a medicament useful in treating cancer, said cancer characterized by expression of at least one of EGFR, erbB-2 or erbB-4. In another embodiment, there is provided use of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof in the preparation of a medicament useful in treating cancer, said cancer characterized by expression of at least one of EGFR, erbB-2 or erbB-4. In a further embodiment, there is provided use of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof in the preparation of a medicament useful in treating cancer, said cancer characterized by expression of at least one of EGFR, erbB-2 or erbB-4. In another embodiment, there is provided use of a salt of a compound of formula (I) wherein said salt is a hydrobromide or tartrate salt in non-solvated form, solvated form, or a mixture of non-solvated or solvated forms thereof in the preparation of a medicament useful in treating cancer, said cancer characterized by expression of at least one of EGFR, erbB-2 or erbB-4. In a further embodiment, there is provided use of a salt of a compound of formula (I) wherein said salt is a fumarate, benzensulfonate, salicylate, succinate, or ethanedisulfonate salt in non-solvated form, solvated form, or a mixture of non-solvated or solvated forms thereof in the preparation of a medicament useful in treating cancer, said cancer characterized by expression of at least one of EGFR, erbB-2 or erbB-4.

In one embodiment, there is provided use of a salt of a compound of formula (I) wherein said salt is a fumarate, methanesulfonate, ethanesulfonate, lactate, benzenesulfonate, malate, maleate, benzoate, hydrobromide, tartrate, citrate, salicylate, succinate, or ethanedisulfonate salt in non-solvated form, solvated form, or a mixture of non-solvated or solvated forms thereof in the preparation of a medicament useful in treating cancer, said cancer characterized by expression of at least two of EGFR, erbB-2 or erbB-4. In another embodiment, there is provided use of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof in the preparation of a medicament useful in treating cancer, said cancer characterized by expression of at least two of EGFR, erbB-2 or erbB-4. In a further embodiment, there is provided use of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof in the preparation of a medicament useful in treating cancer, said cancer characterized by expression of at least two of EGFR, erbB-2 or erbB-4. In another embodiment, there is provided use of a salt of a compound of formula (I) wherein said salt is a hydrobromide or tartrate salt in non-solvated form, solvated form, or a mixture of non-solvated or solvated forms thereof in the preparation of a medicament useful in treating cancer, said cancer characterized by expression of at least two of EGFR, erbB-2 or erbB-4. In a further embodiment, there is provided use of a salt of a compound of formula (I) wherein said salt is a fumarate, benzensulfonate, salicylate, succinate, or ethanedisulfonate salt in non-solvated form, solvated form, or a mixture of non-solvated or solvated forms thereof in the preparation of a medicament useful in treating cancer, said cancer characterized by expression of at least two of EGFR, erbB-2 or erbB-4.

In one embodiment, of each of the preceding methods of treatment and/or uses the mammal is a human.

A therapeutically effective amount of a salt of a compound of formula (I) in non-solvated form, solvated form, or a mixture of non-solvated or solvated forms thereof will depend on a number of factors including, but not limited to, the age and weight of the mammal, the precise disorder requiring treatment and its severity, the nature of the formulation, and the route of administration, and will ultimately be at the discretion of the attendant physician or veternarian.

The following examples are intended for illustration only and are not intended to limit the scope of the invention in any way. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| N (Normal) | Kg (kilogram) |
| i. v. (intravenous); | Hz (Hertz); |
| MHz (megahertz); | mol (moles); |
| mmol (millimoles); | RT (room temperature); |
| min (minutes); | h (hours); |
| mp (melting point); | TLC (thin layer chromatography); |
| Tᵣ (retention time); | RP (reverse phase); |
| THF (tetrahydrofuran); | DMSO (dimethylsulfoxide); |
| EtOAc (ethyl acetate); | DME (1,2-dimethoxyethane); |
| DCM (dichloromethane); | DCE (dichloroethane); |
| DMF (N,N-dimethylformamide); | HOAc (acetic acid); |

Unless otherwise indicated, all temperatures are expressed in ° C. (degrees Centigrade). All reactions conducted under an inert atmosphere at room temperature unless otherwise noted.

The X-Ray Powder Diffraction (XRPD) analysis shown in the Figures were performed on a Phillips X′ pert Pro powder diffractometer, Model PW3040/60, serial number DY1379 using an X′Celerator detector. The acquisition conditions were; radiation: Cu Kα, generator tension: 45 kV, generator current: 40 mA, start angle: 2.0 °2θ, end angle: 40.0 °2θ, step size: 0.0167 °2θ, time per step: 31.75 seconds. The sample was prepared using silicon wafer technique. The 20 or so most intense peaks plus low angle peaks have been included in the preceding Tables I-XVI.

The Infrared (IR) analyses were performed on a Perkin Elmer infrared spectrometer, model Spectrum One, using a diamond ATR attachment. The acquisition conditions were; number of scans: 16, resolution: 2 cm⁻¹.

The Raman analyses were performed on a Thermo Nicolet Nexus FT-Raman module with a Nexus spectrometer. The sample was placed into an NMR tube for analysis using a 1064 nm excitation laser with power output at the sample of 0.3 W. The acquisition conditions were; number of scans: 120, resolution: 4 cm⁻¹.

The free base of the compound of formula (I) is GW572016X whose generic name is lapatanib and whose chemical name is N-{3-Chloro-4-[(3-fluorobenzyl)oxy]phenyl}-6-[5-({[2-(methane sulphonyl)ethyl]amino}methyl)-2-furyl]-4-quinazolinamine.

GW572016X was prepared according to methods similar to those disclosed herein.

### Example 1

### Preparation of Lapatinib Dimesylate Dihydrate

GW572016X (0.5 g) was heated to reflux in methanol (20 ml). Methanesulfonic acid (0.12 ml) was added to the hot suspension and the solid dissolved. The heat source was removed and crystals were precipitated in less than 1 minute. The resulting suspension was allowed to cool and stirred at ambient temperature for a further 1 hour. The product was filtered, washed with methanol and dried under vacuum at 50° C. for 3 hours to give the title salt and allowed to age exposed to the atmosphere for 3 days. (0.62 g, 89.1% yield) An X-ray powder diffraction pattern and an infrared spectrum were obtained and are depicted in FIG. 1(a) and FIG. 1(b) respectively.

### Example 2

### Preparation of Lapatinib Dimesylate Monohydrate

(a) GW572016X (1.0 g) was heated in a mixture of acetone (12 ml) and water (3 ml) so that the solid dissolved. When the temperature had reached 55° C. methanesulfonic acid (0.24 ml) was added in one portion. The solution was allowed to cool gradually and when the temperature had dropped to 42° C. crystals began to precipitate. The resulting suspension was stirred at ambient temperature overnight and the product then was filtered and dried under vacuum at 45° C. for 24 hours to give the title salt. (1.22 g, 90% yield). An X-ray powder diffraction pattern and an infrared spectrum of the salt were obtained and are depicted in FIG. 2(a) and FIG. 2(b) respectively.

(b) The dimesylate monohydrate salt was also prepared as follows. Methanesulfonic acid (0.24 ml) was added to a solution of GW572016X (1.0 g) in a mixture of tetrahydrofuran (9 ml) and water (1 ml). The solution was stirred at ambient temperature and crystals began to precipitate in approximately 5 minutes and amassed. Additional solution of 90:10 tetrahydrofuran:water (10 ml) was added to mobilize and the resulting suspension was stirred for 2 hours. The product was filtered, washed with tetrahydrofuran and dried under vacuum at 45° C. to give the title salt.

### Example 3

### Preparation of Lapatinib Dimesylate

GW572016X (0.5 g) was heated in propan-1-ol (20 ml) until the solid dissolved. Methanesulfonic acid (0.12 ml) was added to the hot solution and crystals were precipitated and rapidly amassed. The thick suspension was allowed to cool and was stirred at ambient temperature for a further 1 hour. The product was filtered, washed with propan-1-ol and dried under vacuum at 50° C. then 75° C. for several hours to give the title salt. (0.62 g, 93.2% yield) The product was allowed to age exposed to the atmosphere for 3 days. An X-ray powder diffraction pattern and an infrared spectrum were obtained and are depicted in FIG. 3(a) and FIG. 3(b) respectively.

### Example 4

### Preparation of Lapatinib Monomesylate

(a) Methanesulfonic acid (0.12 ml) was added to a solution of GW572016X (1.0 g) in a mixture of tetrahydrofuran (9 ml) and water (1 ml). The solution was stirred vigorously at ambient temperature and crystals began to form around the sides of the flask above the solvent line. Within 2 hours the solution began to turn cloudy and the mixture was left to stand at ambient temperature in the stoppered flask for 3 days. The product was filtered, washed with tetrahydrofuran and dried under vacuum at 65° C. for 4 hours to give the title salt. (0.6 g, 51.5% yield) An X-ray powder diffraction pattern and an infrared spectrum were obtained and are depicted in FIG. 4(a) and FIG. 4(b) respectively.

(b) Alternatively GW572016X (3.0 g) was heated in a mixture of THF (27 ml) and water (3 ml) to 55° C. Methanesulfonic acid (0.36 ml) was added at this temperature and the solution was allowed to cool to 50° C. The solution was seeded at this temperature with the product from example 4a (2% w/w 60 mg) and crystals began to precipitate. The resulting suspension was allowed to cool, stirred at ambient temperature for 3 hours and left to stand for 18 hours. Finally the product was filtered, washed with THF, and dried under vacuum at 50° C. for 6 hours to give the title salt.

(c) In another alternative procedure GW572016X (3.0 g) was heated in a mixture of acetone (36 ml) and water (9 ml) to 55° C. so that the solid dissolved. Methanesulfonic acid (0.36 ml) was added at this temperature and the solution was allowed to cool to 50° C. The solution was seeded at this temperature with the product from example 4b (2% w/w 60 mg) and crystals began to precipitate. The resulting suspension was allowed to cool and stirred at ambient temperature for 6 hours. Finally the product was filtered, washed with acetone and dried under vacuum at 65° C. for 24 hours to give the title salt. (3.0 g, 85.8% yield)

### Example 5

### Preparation of Lapatinib Diesylate

Ethanesulfonic acid (1.90 g, 17.3 mmol) was added to a stirred suspension of GW572016X (5.0 g, 8.6 mmol) in methanol (100 ml) at 21° C. An additional amount of methanol (50 ml) was added. The reaction was heated at 60° C. for 30 minutes, then cooled to 21° C. and left for 26 hours. The bright yellow solid was collected by filtration and washed with methanol (50 ml) then dried under vacuum for 2 hours 50 minutes at 45° C. to give the title salt. (6.16 g, 89.3% yield) An X-ray powder diffraction pattern and an infrared spectrum were obtained and are depicted in FIG. 5(a) and FIG. 5(b) respectively.

### Example 6

### Preparation of Lapatinib Monoesylate

Ethanesulfonic acid (947.6 mg, 8.6 mmol) was added to a stirred suspension of GW572016X (5.0 g, 8.6 mmol) in a mixture of acetone (75 ml) and water (5 ml) at 21° C. The reaction was heated at reflux for 30 minutes then cooled to 21° C. and stirred for 18 hours to give the title salt. The yellow solid was collected by filtration, washed with acetone (75 ml) then dried under vacuum at 40° C. for 64 hours. (5.04 g, 84.7% yield). An X-ray powder diffraction pattern and infrared spectrum were obtained and are depicted in FIG. 6(a) and FIG. 6(b) respectively.

### Example 7

### Preparation of Lapatinib Dimaleate Anhydrate

GW572016X (3.0 g 5.16 mmol) was heated to reflux in a mixture of ethanol (81 ml) and water (9 ml) so that the solid dissolved. Maleic acid (1.2 g 10.34 mmol) was added to the hot solution and crystals were precipitated in less than 1 minute and amassed. The cake was broken up with a spatula and the suspension was allowed to cool, stirred for a further 2 hours at ambient temperature then left to stand overnight. Finally the product was filtered and dried under vacuum at 45° C. for 24 hours to give the title salt. (3.75 g, 89% yield). An X-ray powder diffraction pattern and an infrared spectrum were obtained and are depicted in FIG. 7(a) and FIG. 7(b) respectively.

### Example 8

### Preparation of Lapatinib Dimaleate Monohydrate

GW572016X (3.0 g) was heated to greater than 50° C. in a mixture of acetone (54 ml) and water (6 ml) so that the solid dissolved. Maleic acid (1.2 g) was added to the hot solution and crystals were precipitated rapidly and amassed. More 90:10 acetone:water (30 ml) was added to mobilize and the suspension was stirred at reflux for 1 hour. The suspension was then allowed to cool and was stirred for a further 2 hours at ambient temperature. Finally the product was filtered, washed with acetone and dried under vacuum at 45° C. for 3 hours to give the title salt. (3.7 g, 88% yield). An X-ray powder diffraction pattern and an infrared spectrum were obtained and are depicted in FIGS. 8(a) and 8(b) respectively.

### Example 9

### Preparation of Lapatinib Di-L-Lactate

(a) GW572016X (0.5 g) was heated to reflux in ethyl acetate (30 ml). L-lactic acid (0.17 g) was added and the cloudy mixture cleared. The solution was stirred at reflux for 15 minutes then the solution was filtered hot to remove any residual acid. The filtrate was allowed to cool in an open flask and crystals were precipitated. The suspension was left to stand at ambient temperature overnight. The product was filtered, washed with ethyl acetate and dried under vacuum at 40° C. for 5 hours to give the title salt.

(b) Alternatively, GW572016X (3.0 g) was heated to reflux in ethyl acetate (30 ml). L-lactic acid (0.95 g) was added to the hot suspension and the solid dissolved. The clear solution was stirred at reflux for 15 minutes then allowed to cool. Crystals were precipitated after 30 minutes and the suspension was stirred at ambient temperature overnight. The product was filtered, washed with ethyl acetate and dried under vacuum at 50° C. for 6 hours to give the title salt.

(c) Alternatively, GW572016X (3.0 g) was heated to reflux in DCM (120 ml). L-lactic acid (0.95 g) was added to the warm solution which was stirred at reflux for 5 minutes then allowed to cool. The solution turned cloudy and oil separated. After 30 minutes the mixture was seeded with the product from example 9(b). The oil solidified and more solid was precipitated. The suspension was stirred at ambient temperature for 48 hours. The product was filtered and dried under vacuum at 40° C. for 3 hours to give the title salt. (3.18 g, 80.9% yield) An X-ray powder diffraction pattern and an infrared spectrum were obtained and are depicted in FIG. 9(a) and FIG. 9(b) respectively.

### Example 10

### Preparation of Lapatinib Mono-L-Lactate

A mixture of GW572016X (1.0 g) and L-lactic acid (0.2 g) was heated to 60° C. in acetonitrile (20 ml) so that a clear solution was obtained. The solution was allowed to cool and as the temperature dropped below 40° C. crystals began to precipitate. The resulting suspension was stirred at ambient temperature for 1 hour then left to stand overnight. The product was filtered, washed with acetonitrile and dried under vacuum at 40° C. overnight to give the title salt. (0.9 g, 77.9% yield) An X-ray powder diffraction pattern and an infrared spectrum were obtained and are depicted in FIG. 10(a) and FIG. 10(b) respectively.

### Example 11

### Preparation of Lapatinib Mono-Citrate

(a) Citric acid (660 mg, 3.44 mmol) was added to a stirred suspension of GW572016X (1.0 g, 1.72 mmol) in tetrahydrofuran (40 ml) at 21° C. The reaction mixture was heated at 60° C. for 30 minutes, then cooled to 21° C. and stirred for 3 hours at 21° C. The product was collected by filtration, washed with tetrahydrofuran (20 ml) then dried under vacuum for 24 hours at 40° C. to give the title salt. (0.79 g, 59.4% yield).

(b) Alternatively, citric acid (1.67 g, 8.6 mmol) was added to a stirred suspension of GW572016X (5.0 g, 8.6 mmol) in tetrahydrofuran (50 ml) at 21° C. The reaction mixture was heated at 60° C. for 30 minutes, then cooled to 21° C. After 3.5 hours 30 minutes, the solid was collected by filtration, washed with tetrahydrofuran (50 ml). The product was dried under vacuum for 17 hours at 44° C. to give the title salt. (4.17 g, 62.6% yield). An X-ray powder diffraction pattern and an infrared spectrum were obtained and are depicted in FIG. 11(a) and FIG. 11(b) respectively.

### Example 12

### Preparation of Lapatinib Dibenzoate

Benzoic acid (1.26 g, 10.3 mmol) was added to a stirred suspension of GW572016X (3.0 g, 5.16 mmol) in tetrahydrofuran (30 ml) and water (30 ml) at 21° C. The reaction mixture was heated at 60° C. for 30 minutes then cooled to 21° C. The reaction was concentrated under reduced pressure to give a golden yellow gum. n-Hexane (50 ml) was added and heated at 60° C. for ca. 2 hours, then cooled to 21° C. The solid formed was collected by filtration, washed with n-hexane (20 ml) then dried under vacuum for 17 hours at 43° C. to give the title salt (3.96 g, 93% yield). An X-ray powder diffraction pattern and an infrared spectrum were obtained and are depicted in FIG. 12(a) and FIG. 12(b) respectively.

### Example 13

### Preparation of Lapatinib Mono-L-Malate

(a) GW572016X (1.0 g) was heated in a mixture of ethanol (18 ml) and water (2 ml) to 70° C. so that the solid dissolved. L-Malic acid (0.47 g) was added to the hot solution and the solution was stirred at reflux for 5 minutes then allowed to cool. Crystals were precipitated, initially with some oily deposits which later solidified, and the resulting suspension was allowed to cool, stirred at ambient temperature for 3 hours and then left to stand at ambient temperature overnight. The product was filtered, washed with ethanol and dried under vacuum at 45° C. for 6 hours to give the title salt.

(b) GW572016X (3.0 g) was heated in a mixture of IMS (108 ml) and water (12 ml) to reflux so that the solid dissolved. L-Malic acid (0.7 g) was added to the hot solution and the solution was allowed to cool to 40° C. The solution was seeded at this temperature with the product prepared as in example 18a. Crystals began to precipitate and the resulting suspension was allowed to cool and then stirred at ambient temperature overnight. The product was filtered, washed with IMS and dried under vacuum at 50° C. for 24 hours to give the title salt. (3.14 g, 85.1% yield) An X-ray powder diffraction pattern and an infrared spectrum were obtained and are depicted in FIG. 13(a) and FIG. 13(b) respectively.

### Example 14

### Preparation of Lapatinib Monosuccinate

Succinic acid (2.03 g, 17.2 mmol) was added to a stirring solution of GW572016X (5.0 g, 8.6 mmol) in a mixture of tetrahydrofuran (54 ml) and water (6 ml). The reaction solution was heated at 60° C. for 30 minutes then cooled to 21° C. The reaction mixture was stirred at 21° C. for 27 hours 20 minutes. The solid was collected by filtration, washed with tetrahydrofuran (2×50 ml) then dried under vacuum for 18 hours 20 minutes at 45° C. over phosphorus pentoxide to give the title salt (4.66 g, 77.5% yield). An X-ray powder diffraction pattern and an infrared spectrum were obtained and are depicted in FIGS. 14(a) and 14 (b) respectively.

### Example 15

### Preparation of Lapatinib Dibesylate

GW572016X (0.5 g) was heated in acetonitrile (20 ml) until the solid dissolved. Benzenesulfonic acid (0.32 g) was added to the hot solution and crystals were precipitated rapidly. The suspension was allowed to cool gradually and then stirred at ambient temperature for a further 1 hour. The product was filtered, washed with acetonitrile and dried in the vacuum oven at 45° C. for 3 hours to give the title salt. (0.73 g, 94.5% yield) An X-ray powder diffraction pattern and an infrared spectrum were obtained and are depicted in FIG. 15(a) and FIG. 15(b) respectively.

### Example 16

### Preparation of Lapatinib Di-L-Malate

GW572016X (3.0 g) was heated in ethyl acetate (90 ml) to reflux. L-Malic acid (1.52 g) was added to the hot suspension and the mixture was allowed to cool and stirred at ambient temperature overnight. Finally the product was filtered, washed with ethyl acetate and dried under vacuum at 45° C. for 6 hours to give the title salt. (3.71 g, 84.6% yield)

### Example 17

### Preparation of Lapatinib Monofumarate

A mixture of GW572016X (0.5 g) and fumaric acid (0.21 g) was heated to reflux in methanol (30 ml) and stirred at reflux for 2 hours. The mixture was allowed to cool gradually and stirred at ambient temperature for a further 1 hour. The product was filtered and dried under vacuum at 50° C. for 3-4 hours to give the title salt. (0.46 g, 76.7% yield)

### Example 18

### Preparation of Lapatinib Difumarate

GW572016X (0.5 g) was heated in acetonitrile (10 ml) so that the solid dissolved. Fumaric acid (0.2 g) was added to the warm solution and a solid was precipitated which amassed rapidly. More acetonitrile (10 ml) was added to mobilise and the suspension was allowed to cool and left to stand at ambient temperature for 65 hours. The product was filtered, washed with acetonitrile and dried under vacuum at 50° C. for 18 hours then at 70° C. for 5 hours to give the title salt. (0.66 g, 94.3% yield)

### Example 19

### Preparation of Lapatinib Hydrobromide

Hydrobromic acid (1.91 ml of 48%, 17.2 mmol) was added to a stirring clear solution of GW672016X (5.0 g, 8.6 mmol) in a mixture of tetrahydrofuran (54 ml) and water (6 ml). After 5 minutes, an additional amount of tetrahydrofuran (20 ml) was added. The reaction mixture was heated at 60° C. for 30 minutes, then cooled to 21° C. and left at 21° C. for 23 hours. The yellow solid was collected by filtration, washed with tetrahydrofuran (2×50 ml) then dried under vacuum at 46° C. for 3 hours 45 minutes over phosphorus pentoxide to give the title salt. (6.38 g, 99.9% yield)

### Example 20

### Preparation of Lapatinib Hemi-Edisylate

A mixture of GW573016X (1.0 g) and ethanedisulfonic acid monohydrate (0.36 g) was heated to reflux in a mixture of ethanol (36 ml) and water (4 ml) and the resulting thick suspension was stirred at reflux for 1 hour then allowed to cool. The suspension was stirred at ambient temperature for a further 4 hours then the product was filtered, washed with ethanol and dried under vacuum at 45° C. for 18 hours to give the title salt. (0.92 g, 79.1% yield)

### Example 21

### Preparation of Lapatinib Mono-L-Tartrate

GW572016X (1.0 g) was heated in a mixture of acetonitrile (21 ml) and water (9 ml) to 50° C. so that the solid dissolved. L-tartaric acid (0.27 g) was added at this temperature and the solution was allowed to cool gradually. As the temperature dropped to 44° C. crystals began to precipitate. The resulting suspension was stirred at ambient temperature for 18 hours then the product was filtered, washed with acetonitrile and dried under vacuum oven at 45° C. for 3 hours to give the title salt.

### Example 22

### Preparation of Lapatinib Di-L-Tartrate

GW572016X (1.0 g) was heated in a mixture of acetone (18 ml) and water (2 ml) to reflux so that the solid dissolved. L-Tartaric acid (0.52 g) was added at this temperature and crystals were precipitated rapidly. The suspension was stirred at reflux for 5-10 minutes and more 9:1 acetone:water (10 ml) was added to mobilise. The suspension was allowed to cool and stirred at ambient temperature for a further 2 hours. The product was filtered, washed with acetone and dried under vacuum at 45° C. for 18 hours then a further 24 hours at 55° C. to give the title salt. (1.18 g, 77.8% yield)

### Example 23

### Preparation of Lapatinib Salicylate

GW572016X (3.0 g) was heated in a mixture of acetone (42 ml) and water (18 ml) to 55° C. Salicylic acid (0.72 g) was added at this temperature and the solution was allowed to cool and stirred in an open flask at ambient temperature for 48 hours during which time crystals were gradually precipitated. The product was filtered, washed with acetone and dried under vacuum at 45° C. for 24 hours to give the title salt. (2.27 g, 61.1% yield)

Raman spectra were also obtained on compounds as indicated in the Description of the Drawings above and are depicted in the (c) FIGS. 1 through 15 following. The salts corresponding to the Raman spectra were prepared according to methods similar to those described herein.

### Example 24

### Salt Solubility Measurement

Approximately 50-100 mg of each salt was weighed into a vial and water (10 ml) was added. The vial was sealed with the cap and the contents were stirred for 30 minutes. The vials were checked to ensure that solid was still present. If no solid was present, additional salt was added to the vial in 50-100 mg portions until a slurry remained. The contents of the vial were stirred at ambient temperature for 18-20 hours. The flasks were then checked to establish again whether a solid was still present in the flask. Approximately 2-3 mL of liquid from the vials was taken and filtered through a 0.45 μm syringe filter to remove the solid. For the most soluble salts a 1 ml aliquot of the filtered solution was taken accurately with a glass pipette and diluted to volume (10 or 20 mL) with water:acetonitrile (70:30). The concentration of the solution was determined by hplc assay against a commercial grade standard (assumed purity of 100%). Where possible the remaining solid from the vials was recovered by filtration and an infrared analysis was performed to ensure that the remaining solid was consistent with the input material. The pH of the remaining solution was measured. The results are presented in Table XVII.

TABLE XVII

| | | Solubility | | | |
|---|---|---|---|---|---|
| | | as free | Solubility | | |
| | | base # | as salt | | |
| Salt | Comments | (mg/ml) | (mg/ml) | IR | pH |
| Dimesylate | Cloudy | ~15.3 | ~21.3 | No solid | 2.2 |
| dehydrate | solution | | | recovered | |
| Dimesylate | Gum like | 1) ~4.70 | 1) 6.25 | No solid | 2.6 |
| monohydrate | slurry | 2) ~4.36 | 2) 5.80 | recovered | |
| Dimesylate | Clear | >7.35 | >10.01 | Solution | 2.4 |
| | solution* | | | | |
| Monomesylate | Slurry | 0.33 | 0.39 | As input | 3.3 |
| Di-L-lactate | Clear | >10.3 | >13.5 | Solution | 3.2 |
| | solution* | | | | |
| Mono-L-lactate | Solution | 1) ~7.3 | 1) 8.5 | Solution | 3.6 |
| | slightly | 2) ~6.9 | 2) 7.9 | | |
| | cloudy | | | | |
| Diesylate | Slightly | ~19.7 | ~27.2 | Solution | 2.3 |
| | cloudy | | | | |
| | viscous | | | | |
| | solution | | | | |
| Monoesylate | Slurry | 0.52 | 0.62 | As input | 2.2 |
| | | 0.53 | 0.63 | | |
| | | 0.53 | 0.63 | | 3.0 |
| Dimaleate | Slurry | 0.13 | 0.18 | As input | 3.8 |
| | | 0.15 | 0.20 | | |
| | | 0.14 | 0.19 | | 3.8 |
| Dimaleate | Slurry | 0.04 | 0.06 | As input | 3.9 |
| monohydrate | | 0.04 | 0.06 | | |
| Mono | Slurry | 0.05 | 0.06 | As input | 3.9 |
| L-malate | | 0.05 | 0.07 | | |
| | | 0.05 | 0.06 | | 3.6 |
| Di-L-malate | Cloudy | 0.91 | 1.33 | No solid | 3.2 |
| | Solution | | | recovered | |
| Monocitrate | Slurry | 0.01 | 0.01 | As input | 3.7 |
| | | 0.01 | 0.01 | | |
| Edisylate | Slurry | 0.007 | 0.009 | As input | 3.5 |
| Dibesylate | Slurry | <LoD | — | As input | 3.8 |
| Monofumarate | Slurry | <LoD | — | As input | 4.0 |
| Difumarate | Gum like | <LoD | — | Oil | 3.0 |
| | slurry | | | | |
| Ditosylate | Slurry | 0.005 | 0.008 | As input | 3.9 |
| monohydrate | | 0.004 | 0.006 | | |
| | | 0.003 | 0.004 | | |
| Monotartrate | Slurry | 0.01 | 0.02 | As input | 3.3 |
| Ditartrate | Slurry | 0.01 | 0.02 | Oil | 3.6 |
| Succinate | Slurry | <LoD | <LoD | As input | 4.25 |
| Hydrobromide | Slurry | 0.02 | 0.03 | As input | 2.8 |
| Dibenzoate | Slurry | 0.04 | 0.06 | As input | 3.5 |
| Salicylate | Slurry | 0.002 | 0.003 | As input | 3.9 |

*In this instance all available sample was used up
# For the more highly soluble salts indicated (>5 mg/ml) a higher margin of error may be anticipated with respect to the precise values recorded due to factors associated with handling relatively viscous solutions.

### Biological Data

GW572016X has been tested for erbB family protein tyrosine kinase inhibitory activity in substrate phosphorylation assays and cell proliferation assays. See International Patent Application PCT/EP99/00048 filed Jan. 8, 1999, and published as WO 99/35146 on Jul. 15, 1999. The salts of the present invention may be tested for erbB family protein tyrosine kinase inhibitory activity in substrate phosphorylation assays and cell proliferation assays as follows.

### Substrate Phosphorylation Assay

The substrate phosphorylation assays use baculovirus expressed, recombinant constructs of the intracellular domains of c-erbB-2 and c-erbB-4 that are constitutively active and EGFr isolated from solubilised A431 cell membranes. The method measures the ability of the isolated enzymes to catalyse the transfer of the g-phosphate from ATP onto tyrosine residues in a biotinylated synthetic peptide (Biotin-GluGluGluGluTyrPheGluLeuVal). Substrate phosphorylation was detected following either of the following two procedures:

a.) c-ErbB-2, c-ErbB4 or EGFr were incubated for 30 minutes, at room temperature, with 10 mM MnCl₂, 10 mM ATP, 5 mM peptide, and test compound (diluted from a 5 mM stock in DMSO, final DMSO concentration is 2%) in 40 mM HEPES buffer, pH 7.4. The reaction was stopped by the addition of EDTA (final concentration 0.15 mM) and a sample was transferred to a streptavidin-coated 96-well plate. The plate was washed and the level of phosphotyrosine on the peptide was determined using a Europium-labelled antiphosphotyrosine antibody and quantified with a time-resolved fluorescence technique.

b.) ErbB2 was incubated for 50 minutes at room temperature with 15 mM MnCl2, 2 mM ATP, 0.25 mCi [g-³³P] ATP/well, 5 mM peptide substrate, and test compound (diluted from a 10 mM stock in DMSO, final DMSO concentration is 2%) in 50 mM MOPS pH 7.2. The reaction was terminated by the addition of 200 ml of PBS containing 2.5 mg/ml streptavidin-coated SPA beads (Amersham Inc.), 50 mM ATP, 10 mM EDTA and 0.1% TX-100. The microtitre plates were sealed and SPA beads were allowed to settle for at least six hours. The SPA signal was measured using a Packard Topcount 96-well plate scintillation counter (Packard Instrument Co., Meriden, Conn.).

Results for GW572016X are shown in Table XVIII for EGFR, erbB2, and erbB4 tyrosine kinase inhibition. The structure of the free base (GW572016X) is given.

Table XVIII

| Structure | EGFR | ErbB2 | ErbB4 |
|---|---|---|---|
| 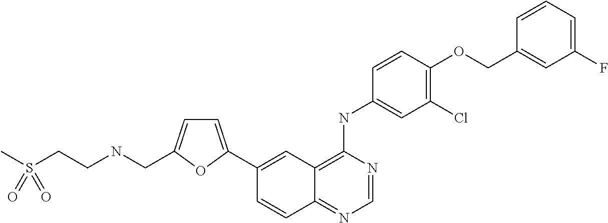 | +++ | +++ | ++ |

| | |
|---|---|
| IC₅₀ values | Symbol |
| <0.10 μM | +++ |
| 0.10-1.0 μM | ++ |
| 1.0-10.0 μM | + |
| >10.0 μM | — |

### Cellular Assays: Methylene Blue Growth Inhibition Assay

Human breast (BT474), head and neck (HN5) and gastric tumor (N87) cell lines and human foreskin Fibroblasts (HFF) were cultured in low glucose DMEM (Life Technologies 12320-032) containing 10% fetal bovine serum (FBS) at 37° C. in a humidified 10% CO₂, 90% air incubator. The SV40 transformed human mammary epithelial cell line HB4a was transfected with either human H-ras cDNA (HB4a r4.2) or the human c-erbB2 cDNA (HB4a c5.2). The HB4a clones were cultured in RPMI containing 10% FBS, insulin (5 mg/ml), hydrocortisone (5 mg/ml), supplemented with the selection agent hygromycin B (50 μg/ml). Cells were harvested using trypsin/EDTA, counted using a haemocytometer, and plated in 100 ml of the appropriate media, at the following densities, in a 96-well tissue culture plate (Falcon 3075): BT474 10,000 cells/well, HN5 3,000 cells/well, N87 10,000 cells/well, HB4a c5.2 3,000 cells/well, HB4a r4.2 3,000 cells/well, HFF 2500 cells/well. The next day, compounds were diluted in DMEM containing 100 mg/ml gentamicin, at twice the final required concentration, from 10 mM stock solutions in DMSO. 100 ml/well of these dilutions were added to the 100 ml of media currently on the cell plates. Medium containing 0.6% DMSO was added to control wells. Compounds diluted in DMEM were added to all cell lines, including the HB4a r4.2 and HB4a c5.2 cell lines. The final concentration of DMSO in all wells was 0.3%. Cells were incubated at 37° C., 10% CO₂ for 3 days. Medium was removed by aspiration. Cell biomass was estimated by staining cells with 1000 per well methylene blue (Sigma M9140, 0.5% in 50:50 ethanol:water), and incubation at room temperature for at least 30 minutes. Stain was removed, and the plates rinsed under a gentle stream of water, and air-dried. To release stain from the cells 1000 of solubilization solution was added (1% N-lauroyl sarcosine, Sodium salt, Sigma L5125, in PBS), and plates were shaken gently for about 30 minutes. Optical density at 620 nM was measured on a microplate reader. Percent inhibition of cell growth was calculated relative to vehicle treated control wells. Concentration of compound that inhibits 50% of cell growth (IC₅₀) was interpolated using nonlinear regression (Levenberg-Marquardt) and the equation, y=Vₘₐₓ(1−(x/(K⁺ x)))+Y2, where “K” was equal to the IC₅₀.

Table XIX illustrates the inhibitory activity of GW572016X as IC₅₀ values in μM against a range of tumor cell lines. Using HFF as a representative human normal cell line, values for cytotoxicity are supplied as IC50 values in micromolar. A measure of selectivity between normal and tumor lines is provided as well.

TABLE XIX

| | N87 | BT474 | HN5 |
|---|---|---|---|
| | IC₅₀ uM | IC₅₀ uM | IC₅₀ uM |
| Compound | Cell | Cell | Cell |
| GW572016X | +++ | +++ | +++ |

| IC₅₀ value | | Symbol |
|---|---|---|
| <5 | μM | +++ |
| 5-25 | μM | ++ |
| 25-50 | μM | + |
| >50 | μM | − |

We claim:

1. A salt of a compound of formula (I), 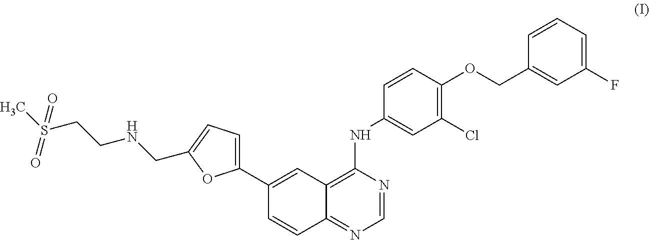
wherein said salt is an ethanesulfonate, methanesulfonate, lactate, malate, maleate, benzoate, or citrate salt in non-solvated form, solvated form, or a mixture of non-solvated or solvated forms thereof.

2. A salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.

3. A pharmaceutical composition including a therapeutically effective amount of a salt of a compound of formula (I) wherein said salt is an ethanesulfonate, methanesulfonate, lactate, malate, maleate, benzoate, or citrate salt in non-solvated form, solvated form, or a mixture of non-solvated or solvated forms thereof.

4. A pharmaceutical composition including a therapeutically effective amount of a salt of a compound of formula (I) wherein said salt is a diesylate, monoesylate, dimesylate, monomesylate, di-L-lactate, mono-L-lactate, di-L-malate, mono-L-malate, dimaleate, dibenzoate, or monocitrate salt in non-solvated form, solvated form, or a mixture of non-solvated or solvated forms thereof.
